SEQUENCE LISTING (50 sequences)

SEQ ID NO: 1 - protein, 148 aa, Artificial
  PLA2-GIB
MKLLVLAVLLTVAAADSGISPRAVWQFRKMIKCVIPGSDPFLEYNNYGCYCGLGGSGTPVDELDKCCQTHDNCYDQAKKLDSCKFLLDNPYTHTYSYSCSGSAITCSSKNKECEAFICNCDRNAAICFSKAPYNKAHKNLDTKKYCQS

SEQ ID NO: 2 - protein, 282 aa, Artificial
  gC1qR
MLPLLRCVPRVLGSSVAGLRAAAPASPFRQLLQPAPRLCTRPFGLLSVRAGSERRPGLLRPRGPCACGCGCGSLHTDGDKAFVDFLSDEIKEERKIQKHKTLPKMSGGWELELNGTEAKLVRKVAGEKITVTFNINNSIPPTFDGEEEPSQGQKVEEQEPELTSTPNFVVEVIKNDDGKKALVLDCHYPEDEVGQEDEAESDIFSIREVSFQSTGESEWKDTNYTLNTDSLDWALYDHLMDFLADRGVDNTFADELVELSTALEHQEYITFLEDLKSFVKSQ

SEQ ID NO: 3 - protein, 344 aa, Artificial
  PLA2-GIB cofactor
AAIGALFLGFLGAAGSTMGAASVTLTVQARLLLSGIVQQQNNLLRAIESQQHMLRLTVWGIKQLQARVLAVERYLKDQQLLGFWGCSGKLICTTTVPWNASWSNKSLDDIWNNMTWMQWEREIDNYTSLIYSLLEKSQTQQEKNEQELLELDKWASLWNWFDITNWLWYIKIFIMIVGGLVGLRIVFAVLSIVNRVRQGYSPLSLQTRPPVPRGPDRPEGIEEEGGERDRDTSGRLVHGFLAIIWVDLRSLFLLSYHHLRDLLLIAARIVELLGRRGWEVLKYWWNLLQYWSQELKSSAVSLLNAAAIAVAEGTDRVIEVLQRAGRAILHIPTRIRQGLERALL

SEQ ID NO: 4 - protein, 15 aa, Artificial
  PLA2-GIB cofactor
AWNAIWINRKYEQID

SEQ ID NO: 5 - protein, 15 aa, Artificial
  PLA2-GIB cofactor
GIAESWPNSLDDSCA

SEQ ID NO: 6 - protein, 15 aa, Artificial
  PLA2-GIB cofactor
GIAESWPNSLDDSCA

SEQ ID NO: 7 - protein, 15 aa, Artificial
  PLA2-GIB cofactor
SFLKSWFNNSLVDIG

SEQ ID NO: 8 - protein, 15 aa, Artificial
  PLA2-GIB cofactor
SGEGGWSNGSLVDIM

SEQ ID NO: 9 - protein, 15 aa, Artificial
  PLA2-GIB cofactor
YELDAASNNSVDDIR

SEQ ID NO: 10 - protein, 15 aa, Artificial
  PLA2-GIB cofactor
AALGKTLVKSLDDIP

SEQ ID NO: 11 - protein, 15 aa, Artificial
  PLA2-GIB cofactor
PWNASWSDKFYENSL

SEQ ID NO: 12 - protein, 15 aa, Artificial
  PLA2-GIB cofactor
AIHASWSNTSYEVID

SEQ ID NO: 13 - protein, 15 aa, Artificial
  PLA2-GIB cofactor
PWNAGWSNARFDELC

SEQ ID NO: 14 - protein, 15 aa, Artificial
  PLA2-GIB cofactor
PWNAIWSAKNTTVDS

SEQ ID NO: 15 - protein, 15 aa, Artificial
  PLA2-GIB cofactor
PWNASWSAAGVGAHA

SEQ ID NO: 16 - protein, 15 aa, Artificial
  PLA2-GIB cofactor
WFNASWKDKSYSTVW

SEQ ID NO: 17 - protein, 15 aa, Artificial
  PLA2-GIB cofactor
PWNASWSVRHISELE

SEQ ID NO: 18 - protein, 15 aa, Artificial
  PLA2-GIB cofactor
PWNASWSYVLDSAWS

SEQ ID NO: 19 - protein, 15 aa, Artificial
  PLA2-GIB cofactor
PWNGSWSNDAWGPGT

SEQ ID NO: 20 - protein, 15 aa, Artificial
  PLA2-GIB cofactor
PWNAGWSNARFDELC

SEQ ID NO: 21 - protein, 15 aa, Artificial
  PLA2-GIB cofactor
PWNAGWSLKSSGKSA

SEQ ID NO: 22 - protein, 15 aa, Artificial
  PLA2-GIB cofactor
PWNAWWSNRSMIADV

SEQ ID NO: 23 - protein, 15 aa, Artificial
  PLA2-GIB cofactor
AWNESWSNKSFHNGA

SEQ ID NO: 24 - protein, 15 aa, Artificial
  PLA2-GIB cofactor
EFNANWSNKFYLYNQ

SEQ ID NO: 25 - protein, 15 aa, Artificial
  PLA2-GIB cofactor
RWNTSWSNWARTERS

SEQ ID NO: 26 - protein, 15 aa, Artificial
  PLA2-GIB cofactor
NWNTSWSNTASGSDS

SEQ ID NO: 27 - protein, 15 aa, Artificial
  PLA2-GIB cofactor
SINAAWSNQSYGFSR

SEQ ID NO: 28 - protein, 15 aa, Artificial
  PLA2-GIB cofactor
NNNAQWSNKEYDKIV

SEQ ID NO: 29 - protein, 15 aa, Artificial
  PLA2-GIB cofactor
HTCASWCNKSLSDIV

SEQ ID NO: 30 - protein, 15 aa, Artificial
  PLA2-GIB cofactor
KDITSWVNKALDAIA

SEQ ID NO: 31 - protein, 15 aa, Artificial
  PLA2-GIB cofactor
GALSSWSNKSLHCCE

SEQ ID NO: 32 - protein, 15 aa, Artificial
  PLA2-GIB cofactor
EKVANWSIKSLGLTV

SEQ ID NO: 33 - protein, 15 aa, Artificial
  PLA2-GIB cofactor
KFIESWSNKSLWLGE

SEQ ID NO: 34 - protein, 15 aa, Artificial
  PLA2-GIB cofactor
FLLYALSNKSLNDIW

SEQ ID NO: 35 - protein, 15 aa, Artificial
  PLA2-GIB cofactor
PTSVSWSNYEQILVG

SEQ ID NO: 36 - protein, 15 aa, Artificial
  PLA2-GIB cofactor
DEQKQWRNKSLEQLW

SEQ ID NO: 37 - protein, 15 aa, Artificial
  PLA2-GIB cofactor
YLGVSWSNKSLLYSY

SEQ ID NO: 38 - protein, 15 aa, Artificial
  PLA2-GIB cofactor
MSKFGLSDKSIEQIH

SEQ ID NO: 39 - protein, 15 aa, Artificial
  PLA2-GIB cofactor
MRYTVESGKSLDDIW

SEQ ID NO: 40 - protein, 15 aa, Artificial
  PLA2-GIB cofactor
FELVCASNKSLEQLA

SEQ ID NO: 41 - protein, 15 aa, Artificial
  PLA2-GIB cofactor
DHDKGLETESLEQIW

SEQ ID NO: 42 - protein, 15 aa, Artificial
  PLA2-GIB cofactor
EPKDFRESATLNQIW

SEQ ID NO: 43 - protein, 191 aa, Artificial
  PLA2-GIB cofactor
  misc_feature (180)..(180): Xaa can be any naturally occurring amino acid
MSTNPKPQRKTKRNTIRRPQDVKFPGGGQIVGGVYLLPRRGPRLGVRATRKTSERSQPRGRRQPIPKARRPEGRTWAQPGYPWPLYGNEGMGWAGWLLSPRGSRPSWGPTDPRRRSRNLGKVIDTLTCGFADLMGYVPLVGAPLGGAARALAHGVRALEDGVNYATGNLPGCSFSISLWXLLSCLTIPASA

SEQ ID NO: 44 - protein, 516 aa, Artificial
  PLA2-GIB cofactor
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 45 - protein, 15 aa, Artificial Sequence
  PEP3 peptide
PWNASWSNKSLDDIW

SEQ ID NO: 46 - protein, 15 aa, Artificial Sequence
  control peptide
PWNATWTQRTLDDIW

SEQ ID NO: 47 - DNA, 25 bp, Artificial Sequence
  Primer
caccgaagtgaccgtgattctaaaa

SEQ ID NO: 48 - DNA, 25 bp, Artificial Sequence
  Primer
aaacttttagaatcacggtcacttc

SEQ ID NO: 49 - DNA, 19 bp, Artificial Sequence
  Primer
tactacagcccttgttctt

SEQ ID NO: 50 - DNA, 18 bp, Artificial Sequence
  Primer
agcacttcctgaaatgtt

(19) United States
(12) Patent Application Publication
(10) Pub. No.: US 2020/0399392 A1
(43) Pub. Date: Dec. 24, 2020

(54) INHIBITORS OF PLA2-G1B COFACTORS FOR TREATING CANCER

(71) Applicant: DIACCURATE, PARIS (FR)

(72) Inventors: JULIEN POTHLICHET, SÈVRES (FR); PHILIPPE POULETTY, PARIS (FR); JACQUES THEZE, PARIS (FR)

(21) Appl. No.: 16/976,088

(22) PCT Filed: Feb. 26, 2019
(86) PCT No.: PCT/EP2019/054687

§ 371 Date: Aug. 27, 2020

(30) Foreign Application Priority Data
Feb. 27, 2018 (EP) 18305207.5

(51) Int. Cl. C07K 16/28; A61K 39/395; C07K 14/005; C12N 7/00; A61K 39/085; A61K 45/06; A61K 39/02

(52) U.S. Cl. CPC C07K 16/2896; A61K 39/3955; C07K 14/005; C12N 7/00; A61K 39/085; C12N 2770/24222; A61K 39/0216; C07K 2317/76; C12N 2740/16222; C12N 2740/16023; C12N 2770/24233; A61K 45/06

(57) ABSTRACT

The present invention relates to novel therapeutic approaches for treating cancer in mammals, particularly in human subjects, using an inhibitor of a PLA2-GIB cofactor.

The present invention relates to novel therapeutic approaches for treating or preventing cancers in mammals, particularly in human subjects. The invention provides therapeutic methods based on the inhibition of a novel mechanism by which various pathogens act in mammals. The invention may be in used in a preventive or curative approach, alone or in combination with other treatments, and is suitable against any cancer.

### Introduction and Background

It has been documented by the inventor that sPLA2-GIB is involved in the inactivation of CD4 T cells in HIV infected patients (see WO2015/097140). It was thus proposed and documented by the inventor that sPLA2-GIB modulators are effective for treating diseases in mammal, e.g., disorders associated with an immune deficiency.

Continuing their research, applicant has now found that the effect of sPLA2-GIB can be mediated and/or amplified by a cofactor present in diseased subjects, and that such cofactor acts through a gC1q receptor at the surface of T cells. In particular, the inventors have now shown that pathogens produce or activate a cofactor which binds to gC1qR, leading to a sensitization of CD4 T cells to inactivation by very low doses of sPLA2-GIB. In patients infected with such pathogens, CD4 T cells become sensitive to inactivation by physiological amounts of sPLA2-GIB, while in non-infected subjects, CD4 T cells remain resistant to inactivation by such physiological concentration of sPLA2-GIB. The inventors have identified such gC1qR-binding cofactors from various pathogens, including viruses or bacteria, such as HIV, HCV or S. aureus. Applicant also verified that said cofactors could sensitize CD4 T cells to inactivation by sPLA2-GIB, and that blocking of such cofactors in vivo could restore or maintain resistance of CD4 T cells to inactivation by sPLA2-GIB. Applicant thus identified a novel general mechanism by which many pathogens induce diseases or pathological conditions in mammals, i.e., by inducing a sensitization of CD4 T cells to inactivation by PLA2-GIB. Such unexpected findings allow applicant to provide novel therapeutic approaches based on a modulation of such cofactor, such as a blockade or inhibition thereof thereby preventing, avoiding or at least reducing the pathogenic effects of many pathogens.

### SUMMARY OF THE INVENTION

It is an object of the invention to provide methods for treating a cancer in a mammalian subject, comprising administering to the subject an inhibitor of a PLA2-GIB cofactor.

Another object of the invention is an inhibitor of a PLA2-GIB cofactor, for use for treating cancer in a mammalian subject.

Another object of the invention relates to the use of an inhibitor of a PLA2-GIB cofactor for the manufacture of a medicament for treating cancer in a mammalian subject.

The inhibitor may be used alone or in combination with any other active agent(s). In particular, the inhibitor may be used in a combination therapy or therapeutic regimen with at least one further anticancer treatment.

The invention may be used in any mammal, particularly in human subjects.

### LEGEND TO THE FIGURES

FIG. 1. Viremic plasma contains a cofactor that causes sensitivity of CD4 T cells to PLA2-GIB activity. A-CD4 T cells purified from 4 healthy donors were exposed or not (w/o GIB) for 30 min to 5 nM or 75 nM of PLA2-GIB (GIB) in PBS BSA 1% buffer (Buffer), 1% of healthy donor plasma (pHD) or viremic patient plasma (pVP) previously depleted with anti-PLA2-GIB antibody to remove endogenous PLA2-GIB activity on CD4 T cells. Then cells were treated with IL-7 for 15 min and the nuclear translocation of pSTAT5 (pSTAT5 NT) was evaluated by confocal microscopy. Results presented the percentage of pSTAT5 NT normalized with the pSTAT5 NT in response to IL-7 in buffer. Statistical analysis the effect of viremic patient plasma on 5 nM of PLA2-GIB was compared to healthy donor plasma using Unpaired t-test. B-Purified CD4 T cells were exposed to 1% of healthy donor plasma (pHD) or viremic patient plasma (pVP) previously depleted with anti-PLA2-GIB antibody and fractionated to separate fraction of molecular weight of more and less than 30 kDa and more and less than 10 kDa or between 30 kDa and 10 kDa.

FIG. 2. AT-2-inactivated HIV-1 particles cause sensitivity of CD4 T cells to PLA2-GIB activity. Purified CD4 T cells were pretreated for 15 min with PBS BSA 1% buffer, HIV-1 AT-2 inactivated particles or similar dilutions of Mock control. HIV-1 particles were used at 5000, 500, 50 and 5 pg of p24/10e⁶ cells which respectively represents multiplicity of infection (MOI) of 1, 0.1, 0.01 and 0.001. Then cells were treated or not for 30 min with 5 nM, 75 nM or 250 nM of PLA2-GIB in PBS BSA 1% as control of PLA2-GIB inhibition conditions or with 5 nM or not of PLA2-GIB with HIV-1 particles or Mock. Then cells were treated with IL-7 for 15 min and the nuclear translocation of pSTAT5 (pSTAT5 NT) was evaluated by confocal microscopy. Results are the percentage of pSTAT5 NT in response to IL-7 with the SEM variation calculated on more than 3 independent fields. **p<0.01 and ***p<0.001 between conditions with GIB relatively to IL-7 treatment without PLA2-GIB. #p<0.05, ###p<0.001 between conditions with increasing amounts of HIV-1 particles with 5 nM of PLA2-GIB. Statistical analyses were performed using unpaired t-test with Welch's correction.

FIG. 3. Recombinant gp41 protein causes sensitivity of CD4 T cells to PLA2-GIB inhibitory activity on pSTAT5 NT in response to IL-7. A-Dose-effect of recombinant gp41 protein on PLA2-GIB activity on pSTAT5 NT response to IL-7. Purified CD4 T cells from healthy donor were pretreated for 15 min with several amounts of gp41 or buffer (PBS BSA 1%), incubated for 30 min with 5 nM of PLA2-GIB (GIB) or not (w/o GIB) and stimulated with IL-7 for 15 min. pSTAT5 NT was analyzed by confocal microscopy. B. Summary of experiments on 3 independent healthy donors of CD4 T cells treated with 0.5 μg/ml of gp41 for 15 min, 30 min with 5 nM of PLA2-GIB (GIB) or not (w/o GIB) and stimulated with IL-7 for 15 min. A and B, results presented the percentage of inhibition of pSTAT5 NT normalized with the pSTAT5 NT in response to IL-7 in buffer. Statistical analysis of the difference of inhibition with gp41 and 5 nM of PLA2-GIB relatively to gp41 alone without PLA2-GIB with unpaired t-test, **means p<0.01.

FIG. 4. Immunodepletion of viremic patient plasma with anti-gp41 antibody abrogates the inhibitory activity of PLA2-GIB on pSTAT5 NT in CD4 T cells (i.e., restores resistance of CD4 T cells to inactivation by PLA2-GIB). Purified CD4 T cells from 3 independent healthy donors were treated in 3 independent experiments for 30 min with PLA2-GIB alone, as positive control of sensitivity to PLA2-GIB, healthy donor (HD) plasma or viremic patient (VP) plasma, previously depleted with anti-gp41 polyclonal (pAb anti-gp41), control polyclonal antibody (pAb ctrl) or treated without antibody (only) and stimulated with IL-7 for 15 min. Results presented the percentage of inhibition of pSTAT5 NT normalized with the pSTAT5 NT in response to IL-7 in buffer for PLA2-GIB or normalized with the same percentage of healthy donor plasma for viremic patient plasma treated samples. ***means that p<0.001 with unpaired t-test for the difference of pSTAT5 NT inhibition with pAb ctrl relatively to pAb anti-gp41 treated viremic plasma.

FIG. 5. PEP3 peptide induces sensitivity to PLA2-GIB inhibitory activity on pSTAT5 NT in CD4 T cells stimulated with IL-7. A-Amino acid sequences of the PEP3 and control (CTL) peptides studied. B-Dose-effect of PEP3 and CTL peptides on PLA2-GIB activity on the percentage of inhibition of pSTAT5 NT response to IL-7. Purified CD4 T cells from healthy donor were pretreated for 15 min with several amounts of PEP3 or CTL peptides or buffer (PBS BSA1%), incubated for 30 min with 5 nM of PLA2-GIB (5 nM G1B) or not (w/o G1B) and stimulated with IL-7 for 15 min. pSTAT5 NT was analyzed by confocal microscopy. C-Summary of experiments on 3 independent healthy donors of CD4 T cells treated with 0.5 μg/ml of PEP3 for 45 min with 5 nM of PLA2-GIB (GIB 5 nM) or not (w/o G1B) and stimulated with IL-7 for 15 min. B and C, results presented the percentage of inhibition of pSTAT5 NT normalized with the pSTAT5 NT in response to IL-7 in buffer. Statistical analysis of the difference of inhibition with PEP3 and 5 nM of PLA2-GIB relatively to PEP3 alone without PLA2-GIB with unpaired t-test, *means p<0.05.

FIG. 6. gC1qR plays a critical role in the cofactor activity of C1q and PEP3 on PLA2-GIB and is involved in viremic patient plasma inhibitory activity. A-C1q has a cofactor activity on PLA2-GIB and 60.11 as well as 74.5.2 antibodies against gC1qR block C1q PLA2-GIB cofactor activity on CD4 T cells. Purified CD4 T cells were preincubated with 60.11, 74.5.2 or mouse control IgG1 (IgG1 ctrl) or without antibody (w/o), treated with 10 μg/ml of C1q without (w/o) or with 5 nM of PLA2-GIB (GIB 5 nM) and pSTAT5 NT response to IL-7 was analyzed. B—The anti-gC1qR 74.5.2 antibody, but not the 60.11 antibody, blocks the PEP3 peptide PLA2-GIB cofactor activity on CD4 T cells. Cells were treated as in A with 0.5 μg/ml of PEP3 without (w/o) or with 5 nM of PLA2-GIB (GIB 5 nM). C—The anti-gC1qR 74.5.2 antibody, but not the 60.11 antibody, decreases inhibition of pSTAT5 NT in CD4 T cells stimulated with IL-7. Cells were pretreated with anti-gC1qR or control antibodies as in A, treated with 1% or 3% viremic patient (pVP) or healthy donor (pHD) plasma for 45 min and pSTAT5 NT response to IL-7 was analyzed. Results in A, B and C are presented as percentage±SEM of inhibition of pSTAT5 NT normalized with percentage of inhibition with IgG1 ctrl and 5 nM GIB with C1q in A or with PEP3 in B and IgG1 ctrl with 1% or 3% of viremic patient plasma in C in one representative experiment. Statistical analyses are the results of unpaired t-test with Welch's correction on at least three independent fields by condition. #p<0.05, ##p<0.01 and ###p<0.001 in each experimental condition with PLA2-GIB vs without PLA2-GIB in A and B or with each percentage of viremic patient plasma vs with the same percentage of healthy donor plasma in C. *p<0.05, **p<0.01 and ***p<0.001 in each experimental condition relatively to cells treated with control IgG1 antibody.

FIG. 7. gp41 increases PLA2-GIB enzymatic activity on CD4 T cells membranes. Purified CD4 T cells labelled with [3H] arachidonic acid were exposed to several concentrations of recombinant gp41 alone or with 63 nM, 200 nM of PLA2-GIB or with PLA2-GIB without gp41 (Medium only). Results are presented as mean cpm/ml±SEM of triplicate of stimulation due to release of [3H] arachidonic acid by PLA2-GIB minus activity in medium alone for each gp41 concentration and are representative of one experiment out of 4 independent experiments with similar results. Statistical analyses are unpaired t-test, *p<0.05, **p<0.01 and ***p<0.001 between experimental condition with gp41 and PLA2-GIB vs PLA2-GIB alone.

FIG. 8. HCV core protein increases PLA2-GIB enzymatic activity on CD4 T cells membranes. A-Dose-effect of HCV core protein on [3H] arachidonic acid release and PLA2-GIB enzymatic activity. Purified CD4 T cells labelled with [3H] arachidonic acid were exposed to several concentrations of HCV core protein alone (HCV core only) or with 63 nM, 200 nM of PLA2-GIB or with PLA2-GIB without HCV core (Buffer only). Results are presented as mean cpm/ml of duplicate of stimulation due to release of [3H] arachidonic acid by PLA2-GIB minus activity in medium with buffer alone for each protein concentration of one experiment. B-HCV core protein increases PLA2-GIB activity. Purified CD4 T cells labelled with [3H] arachidonic acid were exposed to 10 μg/ml of HCV core protein alone (0 nM) or with 63 nM, 200 nM of PLA2-GIB or with PLA2-GIB without HCV core (Buffer eq 10 μg/ml). Results are presented as mean cpm/ml±SEM of three independent experiments with triplicate of stimulation due to release of [3H] arachidonic acid by PLA2-GIB minus activity in medium with buffer alone equivalent to 10 μg/ml of HCV core protein. Statistical analyses are unpaired t-test, ***p<0.001 between experimental conditions with HCV core protein alone or with PLA2-GIB vs medium alone or PLA2-GIB in Buffer, respectively.

FIG. 9. Staphylococcus aureus protein A (SA protein A) increases PLA2-GIB enzymatic activity on CD4 T cells membranes. Purified CD4 T cells labelled with [3H] arachidonic acid were exposed to several concentrations of SA protein A alone (w/o GIB) or with 63 nM, 200 nM of PLA2-GIB or with PLA2-GIB without SA protein A. A-SA protein A increases basal and PLA2-GIB-induced release of [3H] arachidonic acid. Results are presented as mean cpm/ml±SEM from 3 independent experiments with triplicate of stimulation due to release of [3H] arachidonic acid by SA protein A alone or with PLA2-GIB. Statistical analyses are unpaired t-test, ##p<0.01 and ###p<0.001 between experimental conditions with SA protein A alone vs medium alone and *p<0.05, **p<0.01 and ***p<0.001 between experimental conditions with SA protein A with PLA2-GIB vs PLA2-GIB alone. BSA protein A increases PLA2-GIB activity on CD4 T cells. Results are presented as mean cpm/ml±SEM due to PLA2-GIB activity obtained in 3 independent experiments with triplicate of stimulation with SA protein A and PLA2-GIB minus with SA protein A alone or in medium alone. *p<0.05, **p<0.01 and ***p<0.001 between experimental conditions with SA protein A with PLA2-GIB vs PLA2-GIB alone.

FIG. 10. Simplified model of gp41 and other cofactor effect on PLA2-GIB activity on CD4 T cells membranes. Binding of PLA2-GIB cofactor to gC1qR, such as HIV-1 particles, gp41, PEP3, C1q, HCV core or SA protein A, triggers exocytosis of intracellular vesicles. The fusion of these vesicles with plasma membrane changes the lipid composition and causes PLA2-GIB activity on CD4 T cells membranes. As a result of PLA2-GIB activity, membrane fluidity is increased and cytokines receptors are aggregated in abnormal membrane domain resulting in a dramatic decrease of cytokine signaling and anergy of CD4 T cells.

FIG. 11. PEP3 has a cofactor effect on PLA2GIB.

FIG. 12. PEP3 binds gC1qR.

FIG. 13. gC1qR is involved in PEP3 cofactor effect.

FIG. 14. HCV core protein has a cofactor effect on PLA2-GIB.

FIG. 15. Porphyromonas gingivalis has a cofactor effect on PLA2-GIB.

FIG. 16. Plasma from pancreatic cancer patients has a cofactor effect on PLA2GIB.

Table 1. Proteins containing a potential gC1qR binding element that can act as PLA2-GIB cofactors.

Table 2. List of gC1qR ligands that can act as PLA2-GIB cofactors.

Table 3. Proteins from human pathogens containing a potential gC1 qR binding element. This table is derived from Table 1 and lists proteins and peptides from human pathogens that can act as PLA2-GIB cofactors, and associated diseases.

### DETAILED DESCRIPTION OF THE INVENTION

The invention generally relates to novel therapeutic compositions and methods for treating a mammalian subject in need thereof, which comprise administering a treatment that modulates a PLA2-GIB cofactor. The treatment may comprise administering the cofactor itself; or an activator, agonist or mimotope of the cofactor; or an inhibitor or immunogen of the cofactor. Such treatment is preferably performed in a manner (and the treatment is preferably administered in an amount) which modulates, directly or indirectly, an effect of PLA2-GIB on CD4 T cells, typically in a manner which can maintain or restore resistance of CD4 T cells to inactivation by PLA2-GIB in the subject, or which causes sensitization of CD4 T cells to inactivation by PLA2-GIB in the subject.

### Definitions

As used herein, the term “PLA2-GIB” (or “PLA2-G1B”) designates group IB pancreatic phospholipase A2. PLA2-GIB has been identified and cloned from various mammalian species. The human PLA2-GIB protein is disclosed, for instance, in Lambeau and Gelb (2008). The sequence is available on Genbank No. NP_000919.

The amino acid sequence of an exemplary human PLA2-GIB is shown below (SEQ ID NO: 1).

MKLLVLAVLL TVAAADSGIS PRAVWQFRKM IKCVIPGSDP
FLEYNNYGCY CGLGGSGTPV DELDKCCQTH DNCYDQAKKL
DSCKFLLDNP YTHTYSYSCS GSAITCSSKN KECEAFICNC
DRNAAICFSK APYNKAHKNL DTKKYCQS

Amino acids 1 to 15 of SEQ ID NO: 1 (underlined) are a signal sequence, and amino acids 16 to 22 of SEQ ID NO: 1 (in bold) are a propeptide sequence.

Within the context of the invention, the term “PLA2-GIB” designates preferably human PLA2-GIB.

The human PLA2-GIB protein may be present under two distinct forms: a pro form (pro-sPLA2-GIB), which is activated by proteolytic cleavage of a pro-peptide, leading to the mature secreted form (sPLA2-GIB). The term PLA2-GIB includes any form of the protein, such as the pro-form and/or the mature form. Typically, the mature secreted form comprises the sequence of amino acid residues 23-148 of SEQ ID NO: 1, or any natural variants thereof.

Natural variants of a protein include variants resulting e.g., from polymorphism or splicing. Natural variants may also include any protein comprising the sequence of SEQ ID NO: 1, or the sequence of amino acid residues 23-148 of SEQ ID NO: 1, with one or more amino acid substitution(s), addition(s) and/or deletion(s) of one or several (typically 1, 2 or 3) amino acid residues. Variants include naturally-occurring variants having e.g., at least 90% amino acid sequence identity to SEQ ID NO: 1. Particular variants contain not more than 10 amino acid substitution(s), addition(s), and/or deletion(s) of one or several (typically 1, 2 or 3) amino acid residues as compared to SEQ ID NO: 1. Typical naturally-occurring variants retain a biological activity of PLA2-GIB. In this regard, in some embodiments, PLA2-GIB has at least one activity selected from induction of formation of membrane microdomains (MMD) in CD4 T cells from healthy subjects, or rendering CD4 T cells of healthy subjects refractory to interleukin signaling, such as refractory to IL-2 signaling or refractory to IL-7 signaling or refractory to IL-4 signaling. In some embodiments rendering CD4 T cells of healthy subjects refractory to interleukin-7 signaling comprises a reduction of STAT5A and/or B phosphorylation in said cells by at least about 10%, at least about 20%, at least about 30%, or at least about 40%. In some embodiments rendering CD4 T cells of healthy subjects refractory to interleukin-7 signaling comprises reducing the rate of nuclear translocation of phospho-STAT5A and/or phospho-STAT5B by at least about 20%, at least about 30%, at least about 40%, or at least about 50%.

The term “sequence identity” as applied to nucleic acid or protein sequences, refers to the quantification (usually percentage) of nucleotide or amino acid residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997) Nucleic Acids Res 25:3389-3402). BLAST2 may be used in a standardized and reproducible way to insert gaps in one of the sequences in order to optimize alignment and to achieve a more meaningful comparison between them.

The term “inactivation” indicates, in relation to CD4 T cells, that such cells lose at least part of their ability to contribute to the development of an effective immune response. Inactivation may be partial or complete, transient or permanent. Inactivation designates preferably reducing by at least 10%, 20%, 30%, 40%, 50%, 60%, 70%, 80% or more a function of CD4 T cells, particularly pSTAT5 nuclear translocation and/or CD4 T cell's immunostimulatory activity. Typically, inactive CD4 T cells have no effective pSTAT5 nuclear translocation. In a particular embodiment, an inactive CD4 T cell is an anergic CD4 T cell.

The term “resistance” (or “insensitivity”) of CD4 T cells to inactivation by sPLA2-GIB indicates, within the context of this invention, that CD4 T cells are essentially not inactivated in vitro when incubated in the presence of 5 nM of sPLA2-GIB. Resistance indicates, for instance, that CD4 T cells retain an active nuclear translocation of pSTAT5 when incubated in vitro in the presence of 5 nM sPLA2-GIB and interleukin-7. Resistance (or insensitivity) of CD4 T cells to sPLA2-GIB may also indicate that CD4 T cells incubated in vitro with 5 nM PLA2-GIB remain immunologically functional, e.g., do not become anergic.

### Cofactor Effect

The inventors have found that many pathogens act by rendering CD4 T cells sensitive to inactivation by PLA2-GIB. Such mechanism is believed to involve the binding of a molecule of (or induced by) the pathogen to gC1qR at the surface of CD4 T cells, causing sensitization of CD4 T cells to inactivation by physiological concentrations of PLA2-GIB. In particular, analyzing the mechanism of inactivation of CD4 T cells by PLA2-GIB, the inventors discovered that agonists of gC1qR render CD4 T cells sensitive to low doses of PLA2-GIB. As a result, in the presence of such a cofactor and physiological amounts of PLA2-GIB, CD4 T cells become inactive (e.g., anergic), while they remain active in the presence of physiological amounts of PLA2-GIB only. The inventors verified that gC1q, the natural ligand of gC1qR, exhibits such cofactor effect, and that an anti-gC1q antibody can block such cofactor effect. The inventors also surprisingly found that many pathogens, including viruses and cells, actually contain or produce or activate such cofactors that lead to sensitization of CD4 T cells to inactivation by sPLA2-GIB. In particular, the inventors have shown (i) that HCV core protein can bind gC1qR and cause sensitization of CD4 T cells to inactivation by sPLA2-GIB, (ii) that Staphylococcus protein A can bind gC1qR and cause sensitization of CD4 T cells to inactivation by sPLA2-GIB, (iii) that HIV gp41 can bind gC1qR and cause sensitization of CD4 T cells to inactivation by sPLA2-GIB, and (iv) that plasma from cancer patients cause sensitization of CD4 T cells to inactivation by sPLA2-GIB.

Applicant thus identified a novel general mechanism by which many pathogens induce diseases or pathological status, or (at least temporary) immunodeficiency in mammals, i.e., by producing or activating a cofactor which induces a sensitization of CD4 T cells to inactivation by PLA2-GIB. The inventors particularly discovered that PLA2GIB cofactors in cancers, demonstrating that such mechanism is also involved in the occurrence and development of cancers. Such unexpected findings allow applicant to provide novel therapeutic approaches based on the modulation (e.g., blockade or inhibition or stimulation) of said mechanism, thereby preventing, avoiding or at least reducing the pathogenic effects of many pathogens, or inducing an immunosuppression.

It is thus an object of the invention to provide methods and compositions for treating cancer in a mammalian subject, comprising administering to the subject an inhibitor of a PLA2-GIB cofactor.

Another object of the invention relates to an inhibitor of a PLA2-GIB co-factor, for use for treating cancer in a mammalian subject.

It is a further object of the invention to provide methods and compositions for restoring/maintaining resistance of CD4 T cells to inactivation by PLA2-GIB in mammals having a cancer.

The invention also relates to the use of an inhibitor of a PLA2-GIB cofactor, for the manufacture of a medicament for treating cancer in a subject in need thereof.

### PLA2-GIB Cofactors

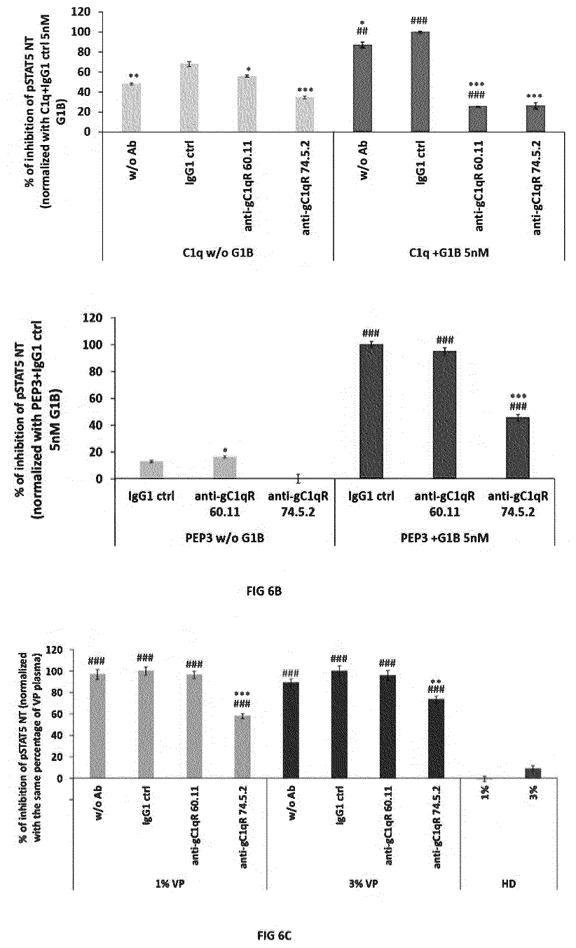

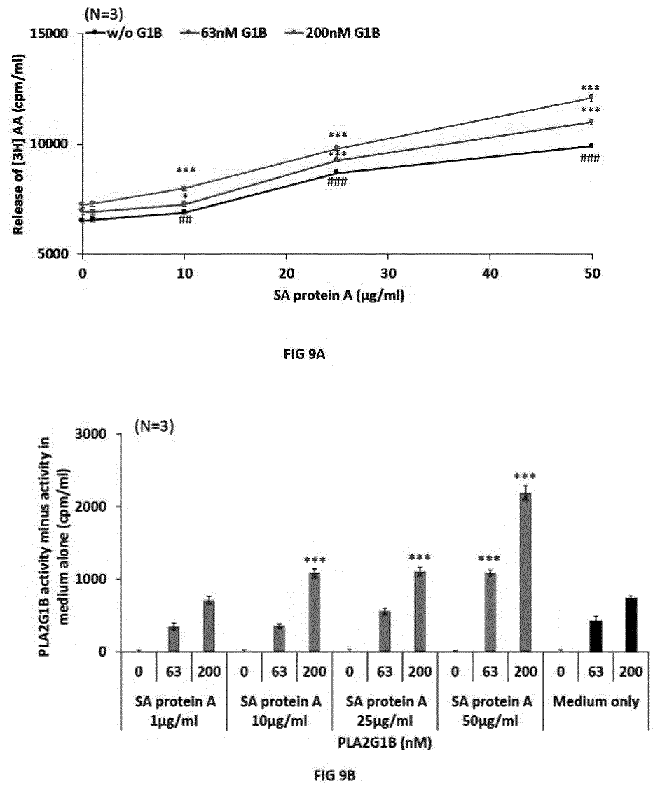

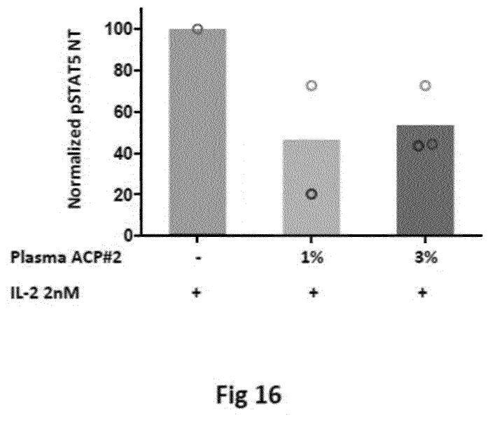

The inventors have surprisingly discovered that many different types of pathogens act as (or produce or activate) a cofactor of PLA2-GIB that, in combination with PLA2-GIB, leads to CD4 T cell inactivation. In particular, as shown FIG. 8, HCV core protein causes sensitization of CD4 T cells to inactivation by low concentrations of sPLA2-GIB. Similarly, as shown FIG. 9, Staphylococcus protein A causes sensitization of CD4 T cells to inactivation by low concentrations of sPLA2-GIB and, as shown FIG. 3-7, HIV gp41 causes sensitization of CD4 T cells to inactivation by low concentrations of sPLA2-GIB. FIG. 15 shows that a peptide from P. gingivalis has a PLA2GIB cofactor effect and FIG. 16 further demonstrates that plasma from cancer patients have a PLA2GIB cofactor effect. The inventors have further discovered that these cofactor molecules are ligands of the gC1qR and that inhibiting their binding to gC1qR also inhibits the cofactor effect (FIGS. 6B and 6C).

The inventors thus identified various molecules produced by pathogens and/or in pathogenic conditions which can bind gC1qR and act as cofactors of sPLA2-GIB.

Within the context of the invention, the term “cofactor” of PLA2-GIB thus designates any molecule or agent which potentiates or amplifies or mediates an effect of PLA2-GIB, particularly an effect of PLA2-GIB on CD4 T cells. Preferred cofactors are molecules which can sensitize CD4 T cells to inactivation by low concentrations of PLA2-GIB.

In a particular embodiment of the invention, the PLA2-GIB cofactor is a ligand of gC1qR. The inventors have indeed demonstrated that ligands of gC1qR at the surface of CD4 T cells act as cofactors of PLA2-GIB, rendering cells more sensitive to inactivation by PLA2-GIB. More particularly, the PLA2-GIB cofactor is an agonist of gC1qR, e.g., can induce signaling through gC1qR, more particularly can induce gC1qR-mediated exocytosis.

In this respect, the inventors have identified various proteins which can act as cofactor of PLA2-GIB, as listed in Tables 1-3. In a particular embodiment of the invention, the PLA2-GIB cofactor is a protein selected from the proteins of Table 1 or 2, or a gC1qR-binding element of such a protein. More particularly, the cofactor may be any protein comprising anyone of SEQ ID NOs: 2-44 or selected from proteins of ID NO: 45-71, more preferably from anyone of SEQ ID NOs: 3, 43, 44 and ID 45-61, even more preferably from anyone of SEQ ID NOs: 3, 43, 44 and ID 45-55, or any fragment or mimotope thereof.

The term “fragment”, in relation to such cofactors, designates preferably a fragment containing a gC1qR-binding element of such a protein, and/or a fragment retaining a capacity of binding gC1qR. Preferred fragments contain at least 5 consecutive amino acid residues, typically between 5 and 100.

In a further particular embodiment, the PLA2-GIB cofactor is a component of a pathogen or a nutrient, preferably a protein or peptide from a pathogen. In a more specific embodiment, the PLA2-GIB cofactor is a viral or bacterial or fungal or parasite protein or peptide. Preferred examples of such cofactors are listed in Tables 2 and 3.

In a specific embodiment, the PLA2-GIB cofactor is HCV core protein, or a fragment or mimotope thereof. In a particular embodiment, the PLA2-GIB cofactor is a protein or peptide comprising or consisting of SEQ ID NO: 43, or a mimotope or fragment thereof.

GenBank: ARQ19013.1
SEQ ID NO: 43
MSTNPKPQRKTKRNTIRRPQDVKFPGGGQIVGGVYLLPRRGPRLGVRATR
KTSERSQPRGRRQPIPKARRPEGRTWAQPGYPWPLYGNEGMGWAGWLLSP
RGSRPSWGPTDPRRRSRNLGKVIDTLTCGFADLMGYVPLVGAPLGGAARA
LAHGVRALEDGVNYATGNLPGCSFSISLWXLLSCLTIPASA

In another specific embodiment, the PLA2-GIB cofactor is Staphylococcus protein A, or a fragment or mimotope thereof. In a particular embodiment, the PLA2-GIB cofactor is a protein or peptide comprising or consisting of SEQ ID NO: 44, or a mimotope or fragment thereof.

NCBI Reference Sequence: YP_498670.1
SEQ ID NO: 44
MKKKNIYSIRKLGVGIASVTLGTLLISGGVTPAANAAQHDEAQQNAFYQV
LNMPNLNADQRNGFIQSLKDDPSQSANVLGEAQKLNDSQAPKADAQQNNF
NKDQQSAFYEILNMPNLNEAQRNGFIQSLKDDPSQSTNVLGEAKKLNESQ
APKADNNFNKEQQNAFYEILNMPNLNEEQRNGFIQSLKDDPSQSANLLSE
AKKLNESQAPKADNKFNKEQQNAFYEILHLPNLNEEQRNGFIQSLKDDPS
QSANLLAEAKKLNDAQAPKADNKFNKEQQNAFYEILHLPNLTEEQRNGFI
QSLKDDPSVSKEILAEAKKLNDAQAPKEEDNNKPGKEDNNKPGKEDNNKP
GKEDNNKPGKEDNNKPGKEDGNKPGKEDNKKPGKEDGNKPGKEDNKKPGK
EDGNKPGKEDGNKPGKEDGNGVHVVKPGDTVNDIAKANGTTADKIAADNK
LADKNMIKPGQELVVDKKQPANHADANKAQALPETGEENPFIGTTVFGGL
SLALGAALLAGRRREL

In another specific embodiment, the PLA2-GIB cofactor is HIV gp41 or rev, or a fragment or mimotope thereof. In a particular embodiment, the PLA2-GIB cofactor is a protein or peptide comprising or consisting of SEQ ID NO: 3 or ID NO: 51, or a fragment or mimotope thereof. Such cofactor is particularly associated with HIV infection.

GenBank reference AAC31817.1
SEQ ID NO: 3
AAIGALFLGFLGAAGSTMGAASVTLTVQARLLLSGIVQQQNNLLRAIESQ
QHMLRLTVWGIKQLQARVLAVERYLKDQQLLGFWGCSGKLICTTTVPWNA
SWSNKSLDDIWNNMTWMQWEREIDNYTSLIYSLLEKSQTQQEKNEQELLE
LDKWASLWNWFDITNWLWYIKIFIMIVGGLVGLRIVFAVLSIVNRVRQGY
SPLSLQTRPPVPRGPDRPEGIEEEGGERDRDTSGRLVHGFLAIIWVDLRS
LFLLSYHHLRDLLLIAARIVELLGRRGWEVLKYWWNLLQYWSQELKSSAV
SLLNAAAIAVAEGTDRVIEVLQRAGRAILHIPTRIRQGLERALL

In another specific embodiment, the PLA2-GIB cofactor is a protein or peptide comprising or consisting of ID NO: 45, or a fragment or mimotope thereof. Such cofactor is particularly associated with EBV infection.

In another specific embodiment, the PLA2-GIB cofactor is a protein or peptide comprising or consisting of ID NO: 46, or a fragment or mimotope thereof. Such cofactor is particularly associated with Adenovirus infection.

In another specific embodiment, the PLA2-GIB cofactor is a protein or peptide comprising or consisting of ID NO: 47, or a fragment or mimotope thereof. Such cofactor is particularly associated with Hantaan virus infection.

In another specific embodiment, the PLA2-GIB cofactor is a protein or peptide comprising or consisting of ID NO: 48, or a fragment or mimotope thereof. Such cofactor is particularly associated with HSV infection.

In another specific embodiment, the PLA2-GIB cofactor is a protein or peptide comprising or consisting of ID NO: 49 or 50, or a fragment or mimotope thereof. Such cofactor is particularly associated with Rubella virus infection.

In another specific embodiment, the PLA2-GIB cofactor is a protein or peptide comprising or consisting of ID NO: 52, or a fragment or mimotope thereof. Such cofactor is particularly associated with L. monocytogenes infection.

In another specific embodiment, the PLA2-GIB cofactor is a protein or peptide comprising or consisting of ID NO: 53, or a fragment or mimotope thereof. Such cofactor is particularly associated with S. pneumoniae infection.

In another specific embodiment, the PLA2-GIB cofactor is a protein or peptide comprising or consisting of ID NO: 54, or a fragment or mimotope thereof. Such cofactor is particularly associated with B. cereus infection.

In another specific embodiment, the PLA2-GIB cofactor is a protein or peptide comprising or consisting of ID NO: 55, or a fragment or mimotope thereof. Such cofactor is particularly associated with Plasmodium falciparum infection.

In another specific embodiment, the PLA2-GIB cofactor is a protein or peptide comprising SEQ ID NO: 7 or 8, or a fragment or mimotope thereof. Such cofactor is particularly associated with P. gingivalis.

In another specific embodiment, the PLA2-GIB cofactor is a protein or peptide comprising SEQ ID NO: 14, or a fragment or mimotope thereof. Such cofactor is particularly associated with P. mirabilis.

In another specific embodiment, the PLA2-GIB cofactor is a protein or peptide comprising SEQ ID NO: 18, or a fragment or mimotope thereof. Such cofactor is particularly associated with L. weilii str.

In another specific embodiment, the PLA2-GIB cofactor is a protein or peptide comprising SEQ ID NO: 28, or a fragment or mimotope thereof. Such cofactor is particularly associated with T. glycolicus.

In another specific embodiment, the PLA2-GIB cofactor is a protein or peptide comprising SEQ ID NO: 29 or 30, or a fragment or mimotope thereof. Such cofactor is particularly associated with B. fragilis.

In another specific embodiment, the PLA2-GIB cofactor is a protein or peptide comprising SEQ ID NO: 33, or a fragment or mimotope thereof. Such cofactor is particularly associated with C. glabrata.

In another specific embodiment, the PLA2-GIB cofactor is a protein or peptide comprising SEQ ID NO: 38, or a fragment or mimotope thereof. Such cofactor is particularly associated with A. actinomycetemcomitans.

In another specific embodiment, the PLA2-GIB cofactor is a protein or peptide comprising SEQ ID NO: 41, or a fragment or mimotope thereof. Such cofactor is particularly associated with P. somerae.

In another specific embodiment, the PLA2-GIB cofactor is a protein or peptide comprising SEQ ID NO: 42, or a fragment or mimotope thereof. Such cofactor is particularly associated with A. aphrophilus.

Further illustrative examples of cofactors are molecules or agents in the plasma of cancer patients, or variants or derivatives thereof, which can exert a cofactor effect on PLA2GIB.

Treatments that Modulate the Cofactor Effect

The invention provides methods and compositions for treating cancer in a subject and/or for restoring/enhancing CD4 T cell activity in subjects having a cancer, using an inhibitor of a PLA2-GIB cofactor.

The term “inhibitor” of a PLA2-GIB cofactor designates, within the context of this invention, any molecule which can inhibit or neutralize or antagonize, directly or indirectly, the expression or activity of a PLA2-GIB cofactor. An inhibitor may thus be a compound which inhibits production or binding to a target of the PLA2-GIB cofactor; or an immunogen of the PLA2-GIB cofactor (which induces anti-cofactor antibodies), or a cytotoxic agent against the cofactor or against a producing-organism.

In a particular embodiment, the term “inhibitor” of a cofactor designates any molecule or treatment which causes (directly or indirectly) an inhibition of the expression or a function of the cofactor, e.g., cofactor binding to gC1qR or cofactor ability to sensitize CD4 T cells to PLA2-GIB. Inhibiting the cofactor designates preferably reducing by at least 10%, 20%, 30%, 40%, 50%, 60%, 70%, 80% or more the expression or a function of the cofactor, as well as completely blocking or suppressing said expression or function. Depending on the situation, the inhibition may be transient, sustained or permanent.

In a particular embodiment, an inhibitor of the cofactor is a gC1qR inhibitor. Indeed, cofactors bind gC1qR as a target receptor. Blocking or reducing or preventing binding of the cofactor to gC1qR using gC1qR inhibitors can affect the cofactor effect. The term “gC1qR inhibitor” designates any molecule or treatment which causes (directly or indirectly) an inhibition of a function of gC1qR, e.g., gC1qR-mediated exocytosis.

gC1qR designates the receptor for complement C1q at the surface of cells, particularly of CD4 T cells, especially the human form of said receptor. gC1qR is also known as C1q binding protein (C1QBP), ASF/SF2-associated protein p32 (SF2P32); Glycoprotein gC1qBP; Hyaluronan-binding protein 1 (HABP1); Mitochondrial matrix protein p32; gC1q-R protein; p33; C1qBP and GC1QBP. The amino acid sequence of the receptor was disclosed in the art. An exemplary amino acid sequence of human gC1qR is reproduced below (SEQ ID NO: 2):

MLPLLRCVPRVLGSSVAGLRAAAPASPFRQLLQPAPRLCTRPFGLLSVRA
GSERRPGLLRPRGPCACGCGCGSLHTDGDKAFVDFLSDEIKEERKIQKHK
TLPKMSGGWELELNGTEAKLVRKVAGEKITVTFNINNSIPPTFDGEEEPS
QGQKVEEQEPELTSTPNFVVEVIKNDDGKKALVLDCHYPEDEVGQEDEAE
SDIFSIREVSFQSTGESEWKDTNYTLNTDSLDWALYDHLMDFLADRGVDN
TFADELVELSTALEHQEYITFLEDLKSFVKSQ

The term gC1qR designates any receptor of SEQ ID NO: 2 (accession number UniProtKB/Swiss-Prot: Q07021.1) above, as well as processed forms and variants thereof. Variants include naturally-occurring variants having e.g., at least 90% amino acid sequence identity to SEQ ID NO: 2.

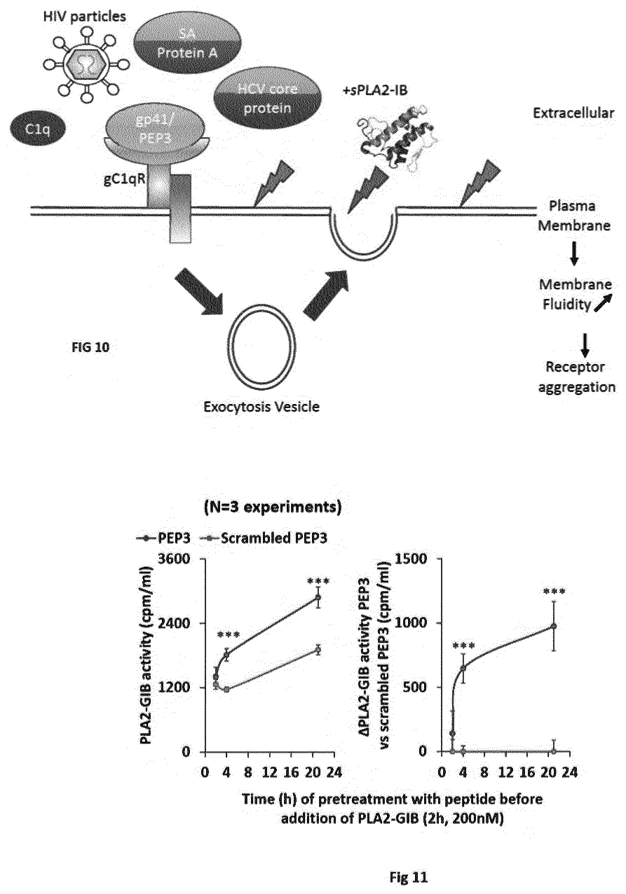

Upon binding of a cofactor, gC1qR triggers a signaling pathway that results in exocytosis of intracellular vesicles. Without being bound by theory, it is believed that the fusion of these vesicles with the cytoplasmic membrane could change the lipid composition and increase sPLA2-GIB activity on CD4 T cells membrane, resulting in an inhibition of phosphoSTAT5 signaling (see FIG. 10). In particular, the fusion of these vesicles with plasma membrane can change the lipid composition and cause sPLA2-GIB activity on CD4 T cells membranes. As a result, membrane fluidity is increased and cytokines receptors are aggregated in abnormal membrane domain, resulting in a dramatic decrease of cytokine signaling, and anergy of CD4 T cells.

The term gC1qR inhibitor thus includes any molecule which binds to gC1qR, or to a partner of gC1qR, and inhibits a function of gC1qR, such as gC1qR-mediated exocytosis.

In another embodiment, the cofactor inhibitor is a molecule which directly inhibits an activity of the cofactor, e.g., which binds the cofactor and/or inhibits binding of the cofactor to its receptor.

Preferred examples of cofactor inhibitors include, for instance, antibodies and variants thereof, synthetic specific ligands, peptides, small drugs, or inhibitory nucleic acids.

### Antibodies

In a first embodiment, a cofactor inhibitor is an antibody or an antibody variant/fragment having essentially the same antigen specificity, or a nucleic acid encoding such an antibody or variant/fragment. The antibody may bind a cofactor, or gC1qR, or a partner of gC1qR, or a gC1qR-binding element thereof, and preferably inhibits a function of the cognate antigen (e.g., gC1qR or the cofactor).

Antibodies can be synthetic, monoclonal, or polyclonal and can be made by techniques well known per se in the art.

The term “antibodies” is meant to include polyclonal antibodies, monoclonal antibodies, fragments thereof, such as F(ab′)2 and Fab fragments, single-chain variable fragments (scFvs), single-domain antibody fragments (VHHs or Nanobodies), bivalent antibody fragments (diabodies), as well as any recombinantly and synthetically produced binding partners, human antibodies or humanized antibodies.

Antibodies are defined to be specifically binding, preferably if they bind to the cognate antigen with a Ka of greater than or equal to about 10⁷ M−1. Affinities of antibodies can be readily determined using conventional techniques, for example those described by Scatchard et al., Ann. N.Y. Acad. Sci., 51:660 (1949).

Polyclonal antibodies can be readily generated from a variety of sources, for example, horses, cows, donkeys, goats, sheep, dogs, chickens, rabbits, mice, hamsters, or rats, using procedures that are well known in the art. In general, a purified immunogen, optionally appropriately conjugated, is administered to the host animal typically through parenteral injection. The immunogenicity of immunogen can be enhanced through the use of an adjuvant, for example, Freund's complete or incomplete adjuvant. Following booster immunizations, small samples of serum are collected and tested for reactivity to the antigen polypeptide. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKeam, and Bechtol (eds.), 1980. For example, the host animals, such as mice, can be injected intraperitoneally at least once and preferably at least twice at about 3 week intervals with isolated and purified immunogen, optionally in the presence of adjuvant. Mouse sera are then assayed by conventional dot blot technique or antibody capture (ABC) to determine which animal is best to fuse. Approximately two to three weeks later, the mice are given an intravenous boost of protein or peptide. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label is added to each well followed by incubation. Positive wells can be subsequently detected. Positive clones can be grown in bulk culture and supernatants are subsequently purified over a Protein A column (Pharmacia). Monoclonal antibodies may also be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

Antigen-binding fragments of antibodies, which can be produced by conventional techniques, are also encompassed by the present invention. Examples of such fragments include, but are not limited to, Fab and F(ab′)2 fragments. Antibody fragments and derivatives produced by genetic engineering techniques are also provided.

The monoclonal antibodies of the invention also include chimeric antibodies, e.g., humanized versions of murine monoclonal antibodies. Such humanized antibodies can be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993). Procedures to generate antibodies transgenically can be found in GB 2,272,440, U.S. Pat. Nos. 5,569,825 and 5,545,806. Antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Publication No. WO 87/02671; Akira, et al. European Patent Application 0184187; Taniguchi, M., European Patent Application 0171496; Morrison et al. European Patent Application 0173494; Neuberger et al. PCT International Publication No. WO 86/01533; Cabilly et al. U.S. Pat. No. 4,816,567; Cabilly et al. European Patent Application 0125023; Better et al., Science 240:1041 1043, 1988; Liu et al., PNAS 84:3439 3443, 1987; Liu et al., J. Immunol. 139:3521 3526, 1987; Sun et al. PNAS 84:214 218, 1987; Nishimura et al., Canc. Res. 47:999 1005, 1987; Wood et al., Nature 314:446 449, 1985; and Shaw et al., J. Natl. Cancer Inst. 80:1553 1559, 1988); Morrison, S. L., Science 229:1202 1207, 1985; Oi et al., BioTechniques 4:214, 1986; Winter U.S. Pat. No. 5,225,539; Jones et al., Nature 321:552 525, 1986; Verhoeyan et al., Science 239:1534, 1988; and Beidler et al., J. Immunol. 141:4053 4060, 1988.

In connection with synthetic and semi-synthetic antibodies, such terms are intended to cover but are not limited to antibody fragments, isotype switched antibodies, humanized antibodies (e.g., mouse-human, human-mouse), hybrids, antibodies having plural specificities, and fully synthetic antibody-like molecules.

Human monoclonal antibodies can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581 597; and Griffths et al. (1993) EMBO J 12:725 734. In addition, a combinatorial library of antibody variable regions can be generated by mutating a known human antibody. For example, a variable region of a human antibody known to bind gC1qR can be mutated by, for example, using randomly altered mutagenized oligonucleotides, to generate a library of mutated variable regions which can then be screened to bind to gC1qR. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457 4461.

Antibodies of the invention may be directed against gC1qR, a gC1qR ligand, or a C1qR partner, and cause an inhibition of signaling mediated by gC1qR. For preparing antibodies of the invention, an immunogen may be used comprising gC1qR, a gC1qR ligand, or a gC1qR partner, or a fragment, variant, or fusion molecule thereof.

Antibodies to gC1qR

Particular antibodies of the invention bind a gC1qR epitope, and/or have been generated by immunization with a polypeptide comprising a gC1qR epitope, selected from the mature gC1qR protein or a fragment of gC1qR comprising at least 8 consecutive amino acid residues thereof. Preferred anti-gC1qR antibodies of the invention bind an epitope of a ligand-binding site within gC1qR, thereby interfering with binding of the ligand. In a particular embodiment, the antibodies bind an epitope comprised between amino acid residues 76-282 of SEQ ID NO: 2, which contain the gC1qR ligand bind site. C1q binding to gC1qR can involve at least three different motifs on gC1qR, namely: amino acid residues 75-96, 190-202 and 144-162 (by reference to SEQ ID NO: 2). HCV core protein binding to gC1qR can involve at least two different motifs on gC1qR, namely: amino acid residues 144-148 and 196-202 (by reference to SEQ ID NO: 2). HIV gp41 binding to gC1qR can involve at least amino acid residues 174-180 on gC1qR (by reference to SEQ ID NO: 2).

It is thus preferred to use an antibody (or variant thereof) which binds an epitope containing at least one amino acid residue contained in one of said epitopes or close to one of said epitopes. Examples of such antibodies include antibody 60.11, which binds to residues 75-96 of gC1qR; as well as antibody 74.5.2, which binds to an epitope with the residues 204 to 218.

Preferred gC1qR inhibitors are therefore monoclonal antibodies against gC1qR, more preferably against an epitope of gC1qR located within amino acid residues 76-282 of the protein (by reference to SEQ ID NO: 2), even more preferably an epitope containing an amino acid residue selected from amino acids 75-96, 144-162, 174-180, and 190-210. Preferred antibodies are neutralizing (or antagonist) antibodies, i.e., they prevent or inhibit or reduce binding of a natural ligand to the receptor and/or signaling through the receptor.

### Antibodies to a PLA2-GIB Cofactor

Other particular inhibitors of the invention are antibodies that bind a PLA2-GIB cofactor and/or have been generated by immunization with a PLA2-GIB cofactor or a fragment thereof, and preferably inhibit at least partially an activity of such cofactor, preferably the binding of such a cofactor to gC1qR.

Particular antibodies of the invention are polyclonal antibodies or monoclonal antibodies, or variants thereof, which bind a protein selected from the proteins listed in Tables 1 and 2, and inhibit at least partially the binding of said protein to gC1qR. Preferred antibodies of the invention are polyclonal antibodies or monoclonal antibodies, or variants thereof, which bind a protein selected from the proteins listed in Tables 2 and 3, and inhibit at least partially the binding of said protein to gC1qR, even more particularly a protein selected from the proteins listed in Table 2, and inhibit at least partially the binding of said protein to gC1qR.

In a particular embodiment, the C1qR inhibitor is an antibody or a variant thereof that binds a protein selected from SEQ ID NOs: 2-44 and ID NO: 45-71, more preferably from SEQ ID NOs: 2, 3, 43, 44 and from ID NO: 45-61, even more preferably from SEQ ID NOs: 3, 43, 44 and ID NO: 45-55, and inhibits at least partially the binding of said protein to gC1qR.

Particular antibodies or variants of the invention bind an epitope within the C1qR ligand contained in (or overlapping with) the gC1qR-binding element or domain of said ligand, typically comprising at least 1 amino acid residue of said ligand that is involved in the binding of said ligand to gC1qR.

In a particular embodiment, the inhibitor is an antibody or variant thereof which binds a protein or peptide comprising SEQ ID NO: 7 or 8. Preferably, such antibody inhibits binding of said protein to a target receptor or cell, particularly to gC1qR.

In another particular embodiment, the inhibitor is an antibody or variant thereof which binds a protein or peptide comprising SEQ ID NO: 14. Preferably, such antibody inhibits binding of said protein to a target receptor or cell, particularly to gC1qR.

In another particular embodiment, the inhibitor is an antibody or variant thereof which binds a protein or peptide comprising SEQ ID NO: 18. Preferably, such antibody inhibits binding of said protein to a target receptor or cell, particularly to gC1qR.

In a particular embodiment, the inhibitor is an antibody or variant thereof which binds a protein or peptide comprising SEQ ID NO: 28. Preferably, such antibody inhibits binding of said protein to a target receptor or cell, particularly to gC1qR.

In a particular embodiment, the inhibitor is an antibody or variant thereof which binds a protein or peptide comprising SEQ ID NO: 29 or 30. Preferably, such antibody inhibits binding of said protein to a target receptor or cell, particularly to gC1qR.

In a particular embodiment, the inhibitor is an antibody or variant thereof which binds a protein or peptide comprising SEQ ID NO: 33. Preferably, such antibody inhibits binding of said protein to a target receptor or cell, particularly to gC1qR.

In a particular embodiment, the inhibitor is an antibody or variant thereof which binds a protein or peptide comprising SEQ ID NO: 38. Preferably, such antibody inhibits binding of said protein to a target receptor or cell, particularly to gC1qR.

In a particular embodiment, the inhibitor is an antibody or variant thereof which binds a protein or peptide comprising SEQ ID NO: 41. Preferably, such antibody inhibits binding of said protein to a target receptor or cell, particularly to gC1qR.

In a particular embodiment, the inhibitor is an antibody or variant thereof which binds a protein or peptide comprising SEQ ID NO: 42. Preferably, such antibody inhibits binding of said protein to a target receptor or cell, particularly to gC1qR.

In a particular embodiment, the inhibitor is an antibody or variant thereof which binds a protein or peptide containing or consisting of SEQ ID NO: 3 or ID45. Preferably, such antibody inhibits binding of said protein to a target receptor or cell, particularly to gC1qR.

In a particular embodiment, the inhibitor is an antibody or variant thereof which binds a protein or peptide containing or consisting of SEQ ID NO: 43. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 51. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 46. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 47. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 48. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 49 or 50. Preferably, such antibody inhibits binding of said protein to a target receptor or cell, particularly to gC1qR.

In a particular embodiment, the inhibitor is an antibody or variant thereof which binds a protein or peptide containing or consisting of SEQ ID NO: 44. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 52. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 53. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 54. Preferably, such antibody inhibits binding of said protein to a target receptor or cell, particularly to gC1qR.

In a particular embodiment, the inhibitor is an antibody or variant thereof which binds a protein or peptide containing or consisting of ID NO: 55. Preferably, such antibody inhibits binding of said protein to a target receptor or cell, particularly to gC1qR.

### Inhibitory Nucleic Acids

In an alternative embodiment, the cofactor inhibitor is an inhibitory nucleic acid. Preferred inhibitory nucleic acids include aptamers which are designed to bind the cofactor, or gC1qR, or a partner of gC1qR, and to inhibit a function thereof.

Other nucleic acids are nucleic acids encoding an antibody as defined above.

### Peptides

In an alternative embodiment, the cofactor inhibitor is a peptide that inhibits a function of the cofactor. The peptide is typically a molecule that selectively binds a cofactor, a gC1qR, or a partner of gC1qR.

Peptides preferably contain from 4 to 30 amino acid residues, and their sequence may be identical to a domain of gC1qR or to a domain of a cofactor (bait peptide), or their sequence may contain a variation as compared to the sequence of a domain of gC1qR or to a domain of a cofactor (peptide antagonist).

Preferred peptides of the invention contain from 4 to 30 consecutive amino acid residues of SEQ ID NO: 2 (gC1qR) or of a cofactor selected from anyone of SEQ ID NOs: 3-71, and may contain at least 1 modification.

The modification may consist of an amino acid substitution. Examples of such substitution includes, without limitation, replacement of a charged or reactive amino acid residue by a more neutral residue such as alanine, or conversely. The modification may alternatively (or in addition) consist of a chemical modification, such as addition of a chemical group to one (or both) ends of the peptide, or to a lateral chain thereof, or to a peptide bond. In this regard, the peptides of the invention can comprise peptide, non-peptide and/or modified peptide bonds. In a particular embodiment, the peptides comprise at least one peptidomimetic bond selected from intercalation of a methylene (—CH₂—) or phosphate (—PO₂—) group, secondary amine (—NH—) or oxygen (—O—), alpha-azapeptides, alpha-alkylpeptides, N-alkylpeptides, phosphonamidates, depsipeptides, hydroxymethylenes, hydroxyethylenes, dihydroxyethylenes, hydroxyethylamines, retro-inverso peptides, methyleneoxy, cetomethylene, esters, phosphinates, phosphinics, or phosphonamides. Also, the peptides may comprise a protected N-ter and/or C-ter function, for example, by acylation, and/or amidation and/or esterification.

Examples of such peptides include, for instance the peptide with amino acid residues 144-162 of SEQ ID NO: 2 (gC1qR) and the peptide with amino acid residues 204-218 of SEQ ID NO: 2 (gC1qR).

Further examples of such peptides of the invention include peptides comprising a sequence of anyone of SEQ ID NOs: 7, 8, 14, 18, 28-30, 33, 38, 41 or 42 with one amino acid substitution, more preferably with at least one amino acid selected from W, I or K replaced with an Alanine.

Further examples of such peptides of the invention include peptides comprising a sequence of anyone of SEQ ID NOs: 7, 8, 14, 18, 28-30, 33, 38, 41 or 42 with one central amino acid deletion.

Further examples of peptides of the invention include peptides comprising the amino acid sequence of SEQ ID NO: 8 with a least one of the following modifications: E3A, W6A, S10A, I14A (for clarity, E3A means that amino acid E in position 3 is replaced with amino acid A).

Further examples of peptides of the invention include peptides comprising the amino acid sequence of SEQ ID NO: 7 with a least one of the following modifications: S1A, K4A, W6A, S10A, I14A (for clarity, S1A means that amino acid S in position 1 is replaced with amino acid A).

The peptides of the invention may be produced by techniques known per se in the art such as chemical, biological, and/or genetic synthesis.

Each of these peptides, in isolated form, represents a particular object of the present invention. The term “isolated”, as used herein, refers to molecules (e.g., nucleic or amino acid) that are removed from a component of their natural environment, isolated or separated, and are at least 60% free, preferably 75% free, and most preferably 90% free from other components with which they are naturally associated. An “isolated” polypeptide (or protein) is for instance a polypeptide separated from a component of its natural environment and, preferably purified to greater than 90% or 95% purity as determined by, for example, electrophoretic (e.g., SDS-PAGE, isoelectric focusing (IEF), capillary electrophoresis) or chromatographic (e.g., ion exchange or reverse phase HPLC) migration. An “isolated” nucleic acid refers to a nucleic acid molecule separated from a component of its natural environment and/or assembled in a different construct (e.g., a vector, expression cassette, recombinant host, etc.).

### Small Drugs

Other inhibitors are small drug inhibitors, such as are hydrocarbon compounds that selectively bind gC1qR or a cofactor.

Small drugs are preferably obtainable by a method comprising: (i) contacting a test compound with a cell expressing gC1qR, (ii) selecting a test compound which binds gC1qR, and (iii) selecting a compound of (ii) which inhibits an activity of gC1qR. Such a method represents a particular object of the invention.

gC1qR Soluble Receptors

In an alternative embodiment, the cofactor inhibitor is a soluble form of gC1qR.

### Cytostatic or Cytotoxic Agents

In another embodiment, the inhibitor is a cytostatic or cytotoxic agent against the PLA2-GIB cofactor or against a prokaryotic or eukaryotic cell or virus expressing a PLA2-GIB cofactor.

Where the cofactor is, or is part of, or is produced by a bacterium, the inhibitor may be an antibiotic against said bacterium. By killing the bacterium, production of the cofactor is avoided. Antibiotic may be any broad-spectrum antibiotic, or an antibiotic with specific spectrum towards the target bacterium. Examples of antibiotics include, but are not limited to, amoxicillin, clarithromycin, cefuroxime, cephalexin ciprofloxacin, clindamycin, doxycycline, metronidazole, terbinafine, levofloxacin, nitrofurantoin, tetracycline, penicillin and azithromycin.

Where the cofactor is, or is part of, or is produced by a eukaryotic cell, the inhibitor may be a cytotoxic agent against said cell. By killing the cell, production of the cofactor is avoided.

Where the cofactor is, or is part of, or is produced by a fungus, the inhibitor may be an antifungal agent. By killing the fungus, production of the cofactor is avoided. Examples of anti-fungal agents, include, but are not limited to, clotrimazole, butenafine, butoconazole, ciclopirox, clioquinol, clioquinol, clotrimazole, econazole, fluconazole, flucytosine, griseofulvin, haloprogin, itraconazole, ketoconazole, miconazole, naftifine, nystatin, oxiconazole, sulconazole, terbinafine, terconazole, tioconazole, and tolnaftate.

Where the cofactor is, or is part of, or is produced by a virus, the inhibitor may be a cytotoxic agent against said virus or an antiviral agent. By killing the virus, production of the cofactor is avoided. Examples of antiviral agents, include, but are not limited to, zidovudine, didanosine, zalcitabine, stavudine, lamivudine, abacavir, tenofovir, nevirapine, delavirdine, efavirenz, saquinavir, ritonavir, indinavir, nelfinavir, saquinavir, amprenavir, and lopinavir.

In another embodiment, the inhibitor of a cofactor is a modulator of the microbiome. Modulation of the composition/diversity of the microbiome can be used to reduce or suppress the production of a cofactor.

In this regard, the invention also provides a method of determining efficacy of a cancer treatment or progression of a cancer in a subject by analyzing the microbiome in said subject, typically before, during and/or after treatment. The method may comprise detecting or measuring the presence, absence or activity of a PLA2GIB cofactor in said microbiome, wherein a reduction in said presence or activity is indicative of an improvement of the subject and/or efficacy of the treatment. More generally, detection or measuring the presence, absence or activity of a PLA2GIB cofactor in any sample from a subject can be used for determining efficacy of a cancer treatment or progression of a cancer in said subject.

### Immunogens

In an alternative (or cumulative) embodiment, inhibition of the cofactor in a subject is obtained by using (e.g., vaccinating or immunizing the subject with) an immunogen of the cofactor. As a result, the subject produces antibodies (or cells) which inhibit the cofactor. In particular, administration(s) of a cofactor immunogen (e.g., any immunogenic portion of a cofactor) can generate antibodies in the treated subject. These antibodies will inhibit the cofactor effect as immunotherapy or a vaccine prophylaxy.

An object of the invention thus resides in a method of vaccinating a subject comprising administering to the subject an immunogen of a PLA2-GIB cofactor.

A further object of the invention relates to an immunogen of a PLA2-GIB cofactor for use to vaccinate a subject in need thereof.

In a particular embodiment, the immunogen of a PLA2-GIB cofactor antigen used for vaccination is an inactivated immunogenic molecule that induces an immune response against the cofactor in a subject. Inactivation may be obtained e.g., by chemically or physically altering the cofactor or by mutating or truncating the protein, or both; and immunogenicity may be obtained as a result of the inactivation and/or by further conjugating the protein to a suitable carrier or hapten, such as KLH, HSA, polylysine, a viral anatoxin, or the like, and/or by polymerization, or the like. The immunogen may thus be chemically or physically modified, e.g., to improve its immunogenicity.

In a preferred embodiment, the immunogen of a PLA2-GIB cofactor of the invention comprises the entire cofactor.

In an alternative embodiment, the immunogen of a PLA2-GIB cofactor comprises a fragment of a cofactor comprising at least 6 consecutive amino acid residues and containing an immunogenic epitope thereof. In a preferred embodiment, the immunogen comprises at least from 6 to 20 amino acid residues. Preferred immunogens of the invention comprise or consist of from 4 to 30 consecutive amino acid residues of a protein selected from anyone of SEQ ID NOs: 2-44 and ID NO: 45-71 (or of a corresponding sequence of a natural variant).

The immunogen may be in various forms such as in free form, polymerized, chemically or physically modified, and/or coupled (i.e., linked) to a carrier molecule. Coupling to a carrier may increase the immunogenicity and (further) suppress the biological activity of the immunogen. In this regard, the carrier molecule may be any carrier molecule or protein conventionally used in immunology such as for instance KLH (Keyhole limpet hemocyanin), ovalbumin, bovine serum albumin (BSA), a viral or bacterial anatoxin such as toxoid tetanos, toxoid diphteric B cholera toxin, mutants thereof such as diphtheria toxin CRM 197, an outer membrane vesicle protein, a polylysine molecule, or a virus like particle (VLP). In a preferred embodiment, the carrier is KLH or CRM197 or a VLP.

Coupling of the immunogen to a carrier may be performed by covalent chemistry using linking chemical groups or reactions, such as for instance glutaraldehyde, biotin, etc. Preferably, the conjugate or the immunogen is submitted to treatment with formaldehyde in order to complete inactivation of the cofactor.

The immunogenicity of the immunogen may be tested by various methods, such as by immunization of a non-human animal grafted with human immune cells, followed by verification of the presence of antibodies, or by sandwich ELISA using human or humanized antibodies. The lack of biological activity may be verified by any of the activity tests described in the application.

In a particular embodiment, the invention relates to an inactivated and immunogenic PLA2-GIB cofactor.

In a further particular embodiment, the invention relates to a PLA2-GIB cofactor protein or a fragment thereof conjugated to a carrier molecule, preferably to KLH.

In a further aspect, the invention relates to a vaccine comprising an immunogen of PLA2-GIB cofactor, a suitable excipient and, optionally, a suitable adjuvant.

A further object of the invention relates to a method for inducing the production of antibodies that neutralize the activity of a PLA2-GIB cofactor in a subject in need thereof, the method comprising administering to said subject an effective amount of a immunogen or vaccine as defined above.

Administration of an immunogen or vaccine of the invention may be by any suitable route, such as by injection, preferably intramuscular, subcutaneous, transdermal, intraveinous or intraarterial; by nasal, oral, mucosal or rectal administration.

### Compositions & Methods of Treatment

The invention also relates to a composition comprising a cofactor or modulator as defined above and, preferably, a pharmaceutically acceptable diluent, excipient or carrier.

A “pharmaceutical composition” refers to a formulation of a compound of the invention (active ingredient) and a medium generally accepted in the art for the delivery of biologically active compounds to the subject in need thereof. Such a carrier includes all pharmaceutically acceptable carriers, diluents, medium or supports therefore. Conventional pharmaceutical practice may be employed to provide suitable formulations or compositions to subjects, for example in unit dosage form.

The compounds or compositions according to the invention may be formulated in the form of ointment, gel, paste, liquid solutions, suspensions, tablets, gelatin capsules, capsules, suppository, powders, nasal drops, or aerosol, preferably in the form of an injectable solution or suspension. For injections, the compounds are generally packaged in the form of liquid suspensions, which may be injected via syringes or perfusions, for example. In this respect, the compounds are generally dissolved in saline, physiological, isotonic or buffered solutions, compatible with pharmaceutical use and known to the person skilled in the art. Thus, the compositions may contain one or more agents or excipients selected from dispersants, solubilizers, stabilizers, preservatives, etc. Agents or excipients that can be used in liquid and/or injectable formulations are notably methylcellulose, hydroxymethylcellulose, carboxymethylcellulose, polysorbate 80, mannitol, gelatin, lactose, vegetable oils, acacia, etc. The carrier can also be selected for example from methyl-beta-cyclodextrin, a polymer of acrylic acid (such as carbopol), a mixture of polyethylene glycol and polypropylene glycol, monoethanolamine and hydroxymethyl cellulose.

The compositions generally comprise an effective amount of an inhibitor of the invention, e.g., an amount that is effective to inhibit directly or indirectly an effect of PLA2-GIB. Inhibitors are typically used in an amount effective to maintain/restore resistance of CD4 T cells to inactivation by PLA2-GIB. Generally, the compositions according to the invention comprise from about 1 μg to 1000 mg of an inhibitor, such as from 0.001-0.01, 0.01-0.1, 0.05-100, 0.05-10, 0.05-5, 0.05-1, 0.1-100, 0.1-1.0, 0.1-5, 1.0-10, 5-10, 10-20, 20-50, and 50-100 mg, for example between 0.05 and 100 mg, preferably between 0.05 and 5 mg, for example 0.05, 0.1, 0.2, 0.3, 0.4, 0.5, 1, 2, 3, 4 or 5 mg. The dosage may be adjusted by the skilled person depending on the agent and the disease.

The compositions of the invention can further comprise one or more additional active compounds, for separate, simultaneous or sequential use. Examples of additional active compounds include, but are not limited to, chemotherapeutic drug, antibiotics, antiparasitic agents, antifungal agents or antiviral agents.

In a particular embodiment, the inhibitor is used in combination with chemotherapy or hormonotherapy.

In another particular embodiment, the inhibitor is used in combination with radiotherapy, ultrasound therapy or nanoparticle therapy.

In another particular embodiment, the inhibitor is used in combination with check-point inhibitors, immunotherapy or anti-cancer vaccines.

In another particular embodiment, the inhibitor is used in combination with an inhibitor of PLA2-GIB.

Examples of PLA2-GIB inhibitors are disclosed for instance in WO2015/097140, WO2017/037041 or in WO2017/060405, which are incorporated therein by reference.

In a particular embodiment, the PLA2-GIB inhibitor is an antibody against PLA2-GIB, particularly a monoclonal antibody against PLA2-GIB, or a derivative or fragment thereof such as a ScFv, nanobody, Fab, bispecific antibody, etc. The antibody or derivative or fragment may be human or humanized.

In a particular embodiment, the method or compositions of the invention use a combination of (i) an inhibitor of a PLA2GIB cofactor and (ii) an antibody against PLA2GIB (or a derivative or fragment thereof). In a further particular embodiment, the inhibitor of a PLA2GIB cofactor in an antibody against the cofactor, or an antibiotic, or an antifungal agent, or an antivirus agent.

In another particular embodiment, the method or compositions of the invention use a combination of (i) an inhibitor of a PLA2GIB cofactor and (ii) an indole-based inhibitor of PLA2GIB (such as 3-(2-amino-1,2-dioxoethyl)-2-ethyl-1-(phenylmethyl)-1H-indol-4-yl)oxy)acetic acid or a pharmaceutically acceptable salt, hydrate, or prodrug thereof, such as a sodium salt thereof (Varespladib)). In a further particular embodiment, the inhibitor of a PLA2GIB cofactor in an antibody against the cofactor, or an antibiotic, or an antifungal agent, or an antivirus agent.

In another particular embodiment, the method or compositions of the invention use a combination of (i) an inhibitor of a PLA2GIB cofactor and (ii) a pentapeptide inhibitor of PLA2GIB (such as a cyclic peptide selected from FLSYK, FLSYR and (2NapA)LS(2NapA)R). In a further particular embodiment, the inhibitor of a PLA2GIB cofactor in an antibody against the cofactor, or an antibiotic, or an antifungal agent, or an antiviral agent.

The invention also relates to a method for preparing a pharmaceutical composition, comprising mixing a cofactor or modulator as previously described and a pharmaceutically acceptable diluent or excipient, and formulating the composition in any suitable form or container (syringe, ampoule, flask, bottle, pouch, etc.).

The invention also relates to a kit comprising (i) a composition comprising a cofactor or modulator as previously described, (ii) at least one container, and optionally (iii) written instructions for using the kit.

The compounds and compositions of the invention may be used to treat a variety of diseases, such as infectious diseases and diseases related to an inappropriate (e.g., defective or improper) immune response, particularly to an inappropriate CD4 T cell activity, as well as any disease where an increased immunity may ameliorate the subject condition. These diseases are sometime referred to as “immune disorders” in the present application. This includes immunodefective situations (e.g., caused by viral infection, pathogenic infection, cancer, etc.), autoimmune diseases, grafts, diabetes, inflammatory diseases, cancers, allergies, asthma, psoriasis, urticaria, eczema and the like.

In a particular embodiment, the invention is directed to methods for stimulating an immune response in a subject in need thereof, comprising administering a cofactor inhibitor or immunogen to said subject.

In another particular embodiment, the invention is directed to methods for treating an immunodeficiency or an associated disorder in a subject in need thereof, comprising administering a cofactor inhibitor or immunogen to said subject, preferably in an amount effective to maintain/restore resistance of CD4 T cells to inactivation by PLA2-GIB.

Immunodeficiencies and associated disorders designate any condition or pathology characterized by and/or caused by a reduced immune function or response in a subject.

Immunodeficiencies may be caused by e.g., viral infection (e.g., HIV, hepatitis B, hepatitis C, etc.), bacterial infection, cancer, or other pathological conditions. The term “immunodeficiency-associated disorder” therefore designates any disease caused by or associated with an immunodeficiency. The invention is particularly suitable for treating immunodeficiencies related to CD4-T cells, and associated diseases.

The invention particularly relates to methods for treating cancer in a subject comprising administering to the subject a compound that inhibits a PLA2-GIB cofactor. The inventors have shown that PLA2-GIB cofactors exist in plasma of patients having cancer which, together with PLA2-GIB, induce inactivation of immune cells.

In a particular embodiment, the invention relates to methods for treating cancer or neoplasia in a subject in need thereof, comprising administering to the subject a compound that inhibits a PLA2-GIB cofactor.

The invention also relates to a compound that inhibits a PLA2-GIB cofactor for use for treating cancer or neoplasia in a subject in need thereof.

In a particular embodiment, the method of the invention is for preventing cancer or reducing the rate of cancer occurrence in a subject in need thereof, such as a subject at risk of neoplasia or cancer. In this regard, the invention can be used for treating risk factors for cancers, thereby avoiding or reducing the risk/rate of occurrence of a cancer. Such risk factors include, without limitation, oro-, gastro-, and/or intestinal inflammation and infections, such as pancreatitis.

The invention also relates to a compound that inhibits a PLA2-GIB cofactor for use for preventing cancer or reducing the rate of cancer occurrence in a subject in need thereof.

In another particular embodiment, the method of the invention is for reducing the rate of cancer progression in a subject having a cancer.

In another particular embodiment, the invention relates to a compound that inhibits a PLA2-GIB cofactor for use for reducing the rate of cancer progression in a subject having a cancer.

In another particular embodiment, the method of the invention is for reducing or preventing or treating cancer metastasis in a subject having a cancer, or for killing cancer cells.

In another particular embodiment, the invention relates to a compound that inhibits a PLA2-GIB cofactor for use for reducing or preventing or treating cancer metastasis in a subject having a cancer, or for killing cancer cells in a subject having a cancer.

The invention may be used for treating any cancer.

In a particular embodiment, the cancer is a solid cancer.

In a particular embodiment, the method is used for treating a subject having cancer and expressing a PLA2-GIB cofactor. In a preferred embodiment, the method is used for treating cancer in a subject, wherein a PLA2-GIB cofactor or a prokaryotic or eukaryotic cell or virus expressing a PLA2-GIB cofactor is present in said subject.

In another particular embodiment, the method is used for treating a subject having cancer, wherein PLA2-GIB or a PLA2-GIB cofactor is present in the cancer microenvironment or blood.

The invention is also particularly suitable for treating cancers or neoplasia in subjects having a PLA2GIB-related CD4 T cell deficiency.

The invention may be used to treat cancers at any stage of development. In this regard, most solid cancer develop through four stages:

. Stage I. This stage is usually a small cancer or tumor that has not grown deeply into nearby tissues. It also has not spread to the lymph nodes or other parts of the body. It is often called early-stage cancer.

. Stage II and Stage III. In general, these 2 stages indicate larger cancers or tumors that have grown more deeply into nearby tissue. They may have also spread to lymph nodes but not to other parts of the body.

. Stage IV. This stage means that the cancer has spread to other organs or parts of the body. It may also be called advanced or metastatic cancer.

Some cancers also have a stage 0. Stage 0 cancers are still located in the place they started and have not spread to nearby tissues. This stage of cancer is often highly curable, usually by removing the entire tumor with surgery.

The invention may be used for treating tumors or cancers at stage 0, I, II, III or IV.

The invention may be used to prevent or reduce or treat metastasis of a cancer at stage 0, I, II or III.

The invention may be used to reduce the rate of progression of a cancer at stage 0, I, II, III or IV.

The invention may in particular be used for treating solid cancers selected from pancreatic cancer, melanoma, lung, oesophageal or pharyngeal cancer, retinoblastoma, liver, breast, ovary, renal, gastric, duodenum, uterine, cervical, thyroid, bladder, prostate, bone, brain or colorectal cancer.

In a specific embodiment, the method of the invention is for treating pancreatic cancer. Pancreatic cancer is classified according to which part of the pancreas is affected: the part that makes digestive substances cause exocrine cancers, the part that makes insulin and other hormones cause endocrine cancers. Although there are several different types of pancreatic cancer, 95% of cases are due to an exocrine cancer, the pancreatic ductal adenocarcinoma (PDAC).

PDAC is ranked the fourth among the major cause of death due to cancer. PDAC is projected by researchers to become the second-most leading cause of cancer-related death in the US by 2030. Incidence has more than doubled in 30 years and currently increases by 5% annually. The relative survival rate for 5 years is around 5% and surgical operation is the most efficient option for the treatment of PDAC. The limited availability of diagnostic approaches, and surgery as the solely existing curative option with the survival possibility of only 10% of diagnostic patients, increases the dreadfulness of this disease. The poor prognosis of the disease can be explained by the absence of effective biomarkers for screening and early detection, together with the aggressive behavior and resistance to the currently available chemotherapy.

The invention shows PLA2-GIB inhibition can be used to treat pancreatic cancer. The invention represents a new strategy to prevent pancreatic cancer progression and metastasis. The invention may be used with any type/stage of pancreatic cancer, such as pancreatic ductal adenocarcinoma, neuroendocrine tumor, intraductal papillary-mucinous neoplasama, mucinous cystic neoplasm, and serious cystic neoplasm. The invention is particularly suited for treating pancreatic ductal adenocarcimona, at any stage.

The invention is also particularly suited for treating colorectal cancer, lung cancer, as well as fast-growing cancers. Colorectal cancer is one of the most common cancer of all genders. At all stages, the probability of survival at 5 years is about 55%. (Bossard N, 2007). Indeed, in France, Japan, US, Germany, Italy, Spain and the United Kingdom, more than 180 000 new cases of rectal cancer were diagnosed in 2010. Colorectal cancer is classified into four stages: stage I, which is the least advanced and is primarily managed by surgery, stages II and III, for which patients undergo combined radiochemotherapy (RCT), and stage IV, which is a very advanced and metastasized stage. When a patient is diagnosed with locally advanced (stage II or III) colorectal cancer, the patient is typically treated with RCT prior to surgical resection. The invention is suited for treating stage I, II, III and IV colorectal cancer. The invention is particularly suited for treating colorectal cancer at stage II, III or IV.

The invention is also suitable for treating cancer that induce gastrointestinal and metabolic pathologies.

For use in the present invention, the PLA2-GIB cofactor inhibitor may be administered by any suitable route. Preferably, administration is by injection, such as systemic or parenteral injection or perfusion, e.g., intramuscular, intravenous, intraarterial, subcutaneous, intratumoral, etc. Administration is typically repeated, or continuous. In a particular embodiment, the level of PLA2-GIB or PLA2-GIB cofactor in the tumor or in body fluids is measured during the course of treatment to guide therapeutic regimen.

The PLA2-GIB cofactor inhibitor may be used alone, or in combination with further cancer treatment(s).

In a particular embodiment, the invention relates to a method for treating cancer in a subject comprising administering to the subject having a cancer a compound that inhibits a PLA2-GIB cofactor in combination with chemotherapy or hormonotherapy.

In another particular embodiment, the invention relates to a method for treating cancer in a subject comprising administering to the subject having a cancer a compound that inhibits a PLA2-GIB cofactor in combination with radiotherapy, ultrasound therapy or nanoparticle therapy.

In another particular embodiment, the invention relates to a method for treating cancer in a subject comprising administering to the subject having a cancer a compound that inhibits a PLA2-GIB cofactor in combination with a check-point inhibitor, immunotherapy or an anti-cancer vaccine.

In another particular embodiment, the invention relates to a method for treating cancer in a subject comprising administering to the subject having a cancer a compound that inhibits a PLA2-GIB cofactor in combination with an inhibitor of PLA2-GIB. The inhibitor of PLA2-GIB may be an antagonist thereof, or a vaccine against said PLA2-GIB.

In a “combination” therapy, the active agents may be used simultaneously or sequentially, together or in alternance. Each active agent may be used according to a specific schedule.

In other instances, all active agents may be formulated and/or administered together, such as in a perfusion.

In a further embodiment, the compound is administered prior to, during or after surgery (tumor resection or removal).

As used herein, “treatment” or “treat” refers to clinical intervention in an attempt to alter the natural course of the individual being treated, and can be performed either for preventive or curative purpose. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions and methods of the invention are used to delay development of a disease or disorder or to slow the progression of a disease or disorder.

The duration, dosages and frequency of administering compounds or compositions of the invention may be adapted according to the subject and disease. The treatment may be used alone or in combination with other active ingredients, either simultaneously or separately or sequentially.

A typical regimen comprises a single or repeated administration of an effective amount of a cofactor or modulator over a period of one or several days, up to one year, and including between one week and about six months. It is understood that the dosage of a pharmaceutical compound or composition of the invention administered in vivo will be dependent upon the age, health, sex, and weight of the recipient (subject), kind of concurrent treatment, if any, frequency of treatment, and the nature of the pharmaceutical effect desired. The ranges of effective doses provided herein are not intended to be limiting and represent preferred dose ranges. However, the most preferred dosage will be tailored to the individual subject, as is understood and determinable by one skilled in the relevant arts (see, e.g., Berkowet et al., eds., The Merck Manual, 16^{th} edition, Merck and Co., Rahway, N.J., 1992; Goodmanetna, eds., Goodman and Cilman's The pharmacological Basis of Therapeutics, 10^{th} edition, Pergamon Press, Inc., Elmsford, N.Y., (2001)).

The invention may be used in any mammal, particularly any human.

Further aspects and advantages of the invention will be disclosed in the following experimental section.

### Examples

### Materials and Methods

Recombinant proteins and peptides—Human PLA2-GIB was produced in E. coli (gift Gerard Lambeau, purity >98%) or in CHO-S(purity >98%). HIV-1 gp41 MN recombinant protein was obtained from Antibodies onlines (gp41 MN (565-771Delta642-725), ABIN2129703, lot 93-482, purity >95%), and PEP3 peptide NH2-PWNASWSNKSLDDIW—COOH and control peptide (CTL) NH2-PWNATWTQRTLDDIW—COOH were ordered from Covalab (purity >98%). HP Pg peptide 8 (peptide SEQ ID NO: 8) NH2-SGEGGWSNGSLVDIM-COOH and Scrambled PEP3 NH2-WNWDSKILSDPAWNS—COOH peptides were ordered from Covalab (purity>98%). Complement component C1q from human serum was obtained from Sigma (C1740, purity >95%). HCV core protein was obtained from Prospec (HCV-011, purity >95%) in PBS buffer with 0.002% SDS and the specificity of effect due to HCV core protein was evaluated by comparison with similar dilution of PBS SDS 0.002%). Staphylococcus aureus protein A was obtained from Sigma (P6031).

Generation of gC1qR KO Jurkat E6.1 T cells—The global strategy for the development of Jurkat cells deprived of C1QBP is based on the design of a targeting vector permitting bi-allelic inactivation of C1QBP gene via homologous recombination. Human C1QBP homologous regions isogenic with the Jurkat E6.1 T cell line (ECACC 88042803) has been used. The targeting vector has been synthetized by Genewiz and cloned into the pUC57-Amp vector. The third exon of human C1QBP gene was targeted by introducing a neomycin resistance gene (NeoR) selection cassette, this results in the interruption of the C1QBP open reading frame. The NeoR cassette was cloned using BamHI/NotI restriction sites. The targeting vector has been verified by DNA restriction digestion cut with selected restriction enzymes (APaL1, Drd1, Pvu1, Pvu2, BamH1/NotI, Not1/NcoI, NEB) and target region sequencing. The DNA primers corresponding to C1QBP sgRNA (1828-Crispr_1A: CACC-GAAGTGACCGTGATTCTAAAA and 1828-Crispr_1B: AAAC-TTTTAGAATCACGGTCACTTC) were hybridized and cloned (Quick Ligase-New England Biolabs, NEB) into the pX330 plasmid (Addgene, 42230; Feng Zhang, MIT) using BbsI restriction site (NEB).

The Jurkat cells (5×10⁶) were resuspended in 100 μL of Opti-MEM and 7 μg of CRISPR/Cas9 plasmid and 2.5 μg of targeting vector were added. The cells were electroporated with a Nepa21 electroporator. After cell selection in G418 selective medium, the Jurkat cell clones were prescreened by PCR genotyping. Independent cell clones knocked-out for C1QBP gene were amplified and verified by PCR genotyping and target region sequencing. Our validation pipeline for the independent Jurkat cell clones deficient for C1QBP gene consisted of PCR genotyping. The genomic DNA of gene edited Jurkat cells was isolated by proteinase K treatment and phenol purification. Each cell clone with bi-allelic inactivation of C1QBP gene was confirmed by PCR genotyping and by target region sequencing. PCR amplification was performed with Platinum HiFi Taq (Life technologies) for 2 min at 50° C. with primers 1828_RH5_F: TACTACAGCCCTTGTTCTT and 1828_RH3_R: AGCACTTCCTGAAATGTT. The primers are designed in the C1QBP human locus and out of homologous arms. The WT and mutant allele are distinguished in the same PCR reaction. The wild type and mutant allele give 1146-bp and 2362-bp amplification product, respectively. This PCR genotyping protocol allows the identification of the homozygous Jurkat cell clone knocked-out for both alleles of C1QBP gene. The gene disruption in Jurkat cell line was achieved using CRISPR/Cas9 technology. The three independent homozygous Jurkat cell clones deficient for C1QBP gene were obtained and validated by PCR genotyping and target region sequencing.

Immunoblot detection of gC1qR in Jurkat E6.1 T cells—Western-blot analysis of gC1qR protein expression in WT and gC1qR KO Jurkat E6.1 T cells lysates. Cells were lysed in mammalian protein extraction reagent (M-PER, 11884111, Thermo Scientific) buffer and protein amount were quantified with BCA Protein assay kit on cleared supernatant (23227, Pierce, Thermo Scientific). An equal amount of total proteins was loaded for WT and gC1qR KO Jurkat E6.1 T cells (40 μg) and fractionated by SDS-PAGE on Mini-PROTEAN TGX Stain Free Gels 8-16% (4568104, BIORAD), further electrotransferred, and probed by immunoblotting using a specific antibody against gC1qR (60.11 Santa Cruz at 1:50, 74.5.2 Abcam at 1:1000) or ß-actin (AC-74, Sigma at 1:2000) in PBS-Tween 0.05% BSA 5% at room temperature for 2 h and a goat anti-mouse-IgG-HRP (1:20000, 31430, Invitrogen) in PBS PBS-Tween 0.05% BSA 5% for 1 h. Bound antibodies were detected using ECL immunoblotting detection system (NEL103001EA, PerkinElmer).

gC1qR-peptides binding assay-100 μl of peptide PEP3, Scrambled PEP3 or CTL at 100 μg/ml diluted in carbonate buffer, pH 9.6 (15 mM Na2CO3 and 35 mM NaHCO₃) were coated overnight at +4° C. on Nunc Maxisorp flat-bottom microplate (44-2404-21, Thermofisher Scientific). The unbound protein was removed; the wells washed 2× with TBST (20 mM Tris-HCl pH 7.5, 150 mM NaCl, and 0.05% Tween-20) and the unreacted sites blocked by incubation (30 min, room temp) with 300 μl of 3% BSA in TBST. After washing (2× with TBST), the microtiter plate bound peptides was incubated (2 h, room temp.) with different amount of His-tag-gC1qR ranging from 0 to 3 μg/well in triplicate. After washing (5 times with TBST), 100 μl of anti-His tag-HRP antibody (1:1000; 71840-3, Merck) in 3% BSA in TBST was added per well and incubated for 2 h at room temperature. Microplates wells were then washed (5 times with TBST) and 100 μl of TMB ELISA substrate standard solution (UP664781, Interchim) was added per well. Reaction was stopped with 100 μl per well of a H2SO4 solution at 0.16M and OD at 450 nm was measured on a microplate reader (Tecan Infinite M1000 Pro).

gp41 immunodepletion of viremic patient and healthy donor plasma—1 ml of viremic patient plasma or healthy donor plasma were incubated with 100 μg of goat anti-gp41 polyclonal antibody (PA21719, Fisher) or the control goat polyclonal antibody (preimmune, AB108-C, R&D) in 1.5 ml Eppendorf tubes overnight on a rotor at 4° C. Then 200 μl of Protein G sepharose 4 Fast Flow beads (17-0618-01, GE healthcare), washed three times in PBS BSA 1%, were added each sample for 3 h on a rotor at 4° C. To remove beads, samples were first centrifugated at 400×g for 2 min at 4° C., the supernatant was collected and then centrifuged at 16,100×g for 15 min at 4° C. As the control goat polyclonal antibody initially contained sodium azide it was washed with 5 times with PBS on 10 kDa Amicon to remove sodium azide before proceeding to immunodepletion.

AT-2 inactivated HIV-1 particles—To preserve the conformational and functional integrity of HIV particles, inactivation was done with 2,2-dithiodipyridine (AT-2; 43791, Sigma) on HIV-1 NDK (T-tropic) particles and prepared on PHA-stimulated PBMCs as described in (Rossio et al., J Virol. 1998). 2,2-dithiodipyridine (aldrithiol-2; AT-2) covalently modify the essential zinc fingers in the nucleocapsid (NC) protein of human immunodeficiency virus type 1 (HIV-1). HIV-1 particles were inactivated twice with 300 μM of AT-2 for 1 h at 37° C. in a water bath followed by 2 h on ice.

In parallel the supernatant of PHA-stimulated PBMCs was treated as HIV-1 NDK-infected cells supernatant to serve as Mock control (without HIV-1 particles). Inactivation of HIV particles was confirmed by an undetectable TCID50 in the infectivity assay. HIV particle concentration was determined by anti-HIV-1 gag p24 ELISA assay (HIV-1 Gag p24 Quantikine ELISA Kit, DHP240, R&D systems biotechne). HIV-1 particles were used at 5000, 500, 50 and 5 pg of p24/10e⁶ cells. 5000pg of p24/10e⁶ cells (1754 pg of p24/3.5×10e⁵ cells) that is equivalent to 1 particle by cells (multiplicity of infection, MOI, of 1).

Purification of Human CD4 T-lymphocytes—Venous blood was obtained from healthy volunteers through the EFS (Etablissement Français du Sang, Centre Necker-Cabanel, Paris). CD4 T-cells were purified from whole blood using RosetteSep Human CD4+ T cell Enrichment Cocktail (Stem Cell, 15062). This cocktail contains mouse and rat monoclonal antibodies purified from mouse ascites fluid or hybridoma culture supernatant, by affinity chromatography using protein A or Protein G sepharose. These antibodies are bound in bispecific tetrameric antibody complexes which are directed against cell surface antigens on human hematopoietic cells (CD8, CD16, CD19, CD36, CD56 CD66b, TCRγ/δ) and glycophorin A on red blood cells. The rosetteSep antibody cocktail crosslinks unwanted cells in human whole blood to multiple red blood cells, forming immunorosettes. This increases the density of unwanted cells, such that they pellet along with the free red blood cells when centrifuged over a buoyant density medium such as lymphocytes separation medium (Eurobio, CMSMSL01-01).

Whole blood was incubated with RosetteSep Human CD4+ T cell Enrichment Cocktail at 50 μl/ml for 20 minutes at room temperature under gentle shaking (100 rpm), diluted with an equal volume of PBS+2% foetal bovine serum (FBS) and mixed gently. The diluted samples were centrifuged 20 minutes at 1200×g on top of lymphocytes separation medium. The enriched cells were then collected from the density medium at plasma interface and washed twice with PBS+2% FBS. Cells were subsequently resuspended in RPMI 1640 medium (Lonza) supplemented with 5% FBS, 50 mM HEPES pH 7.4, glutamine, penicillin, streptomycin and fungizone (complete medium), counted with a Moxi Z mini automated cell counter (ORFLO, MXZ000). Cells suspension was adjusted at 7×10⁶ cells/ml and equilibrated at least 2 h at 37° C. in a 5% CO2 humidified atmosphere.

The enriched CD4-T cell population was controlled by flow cytometry on a cytoflex (Beckman coulter). The quiescence of recovered CD4 T-cells was controlled by the low level of IL-2Rα (CD25). CD4 T cells were labeled with anti-Human CD3 eFluor780 (eBioscience, clone UCHT1, 47-0038-42), anti-Human CD25-PE (Biolegend, clone BC96, 302605) and anti-human CD4-PerCP (BD, clone SK3, 345770). The enriched CD4-T cell population contains >95% CD3+CD4+ and less than 8% of CD25+.

PLA2-GIB bioassay on CD4 T cells and labelling of specific proteins for optical microscopy—Equilibrated purified CD4 T-cells were loaded (3.5×10⁵cells/50 μl in complete medium) on poly-L-Lysine-coated (Sigma, P8920) round coverslips (14 mm-diameter, Marienfeld) in 24-well polystyrene plates at 37° C. in a thermo-regulated water and mixed with 50 μl of a suspension in PBS BSA1% containing peptides, recombinant proteins together with recombinant PLA2-GIB or not or containing viremic patient plasma (1 or 3%) or healthy donor plasma. The cells suspension was either pretreated with 40 μl of peptides, recombinant protein or HIV-1 NDK particles or mock dilutions in PBS BSA1% for 15 minutes with subsequent addition of 10 μl PLA2-GIB (5 nM at the end) for 30 minutes or directly treated with 50 μl of dilution in PBS BSA 1% with peptides or recombinant protein together with PLA2-GIB (5 nM at the end) for 45 minutes. Cells were activated for 15 minutes with 2 nM recombinant glycosylated human IL-7 (Accrobio System). Cells supernatant was removed and cells were fixed by addition of 500 μl of a 4% paraformaldehyde solution in PBS (Fisher, PFA 32% Electron Microscopy Science, 15714) for 15 minutes at 37° C. and then permeabilized for 20 min in 500 μl of ice-cold 90% methanol/water solution.

Cells were then rehydrated for 15 min in PBS plus 5% fetal bovine serum (FBS) and then labeled. Thus, slides were washed twice after methanol treatment in PBS and rehydrated for 15 min in PBS supplemented with 5% FBS at room temperature. Slides were labelled with primary antibodies (1/120) in 60 μl of PBS 5% FBS for 1 h, washed in PBS buffer 15 times, 5 times in PBS/FBS buffer and then stained with secondary antibodies (1/300) for 1 h. Slides were washed 5 times in PBS 5% FBS buffer, rinsed 15 times in PBS and then mounted in fresh Prolong Gold Antifade (ThermoFisher Scientific, P36930) mounting medium for confocal microscopy. The primary antibodies used consisted of rabbit anti-pSTAT5 (pY694, 9359, Cell Signalling), mouse anti-CD4 (BD Pharmingen, 555344) and secondary antibodies were Donkey anti-mouse IgG-AF488 (Invitrogen, A21202) and Donkey anti-rabbit IgG-AF555 (Invitrogen, A31572).

Blocking of gC1qR with anti-gC1qR antibodies 60.11 and 74.5.2—Equilibrated purified CD4 T-cells were preincubated for 30 min with anti-gC1qR 60.11 (epitope 75-96, Santa Cruz, sc-23884), 74.5.2 (epitope 204-218, Abcam, ab125132) (Ghebrehiwet B et al., Adv Exp Med Biol. 2013) or control IgG1 (mouse IgG1 control Isotype, eBioscience/Affymetrix, 16-4714) and loaded (3.5×10⁵cells/60 μl in complete medium) on poly-L-Lysine-coated (Sigma, P8920) round coverslips (14 mm-diameter, Marienfeld) in 24-well polystyrene plates at 37° C. in a thermo-regulated water. Cells were further treated for 45 min with C1q (Sigma C1740, purity >95%, 10 μg/ml), PEPS peptide (0.5 μg/ml) with or without PLA2-GIB at 5 nM or viremic patient plasma 1% or 3% in final volume of 100 μl. Then cells were stimulated with IL-7 and treated as described above to analyze pSTAT5 NT by confocal microscopy.

Confocal Microscopy—Images were acquired above the diffraction limit on an inverted laser scanning confocal microscope (LSM700, Zeiss), with an oil-immersion plan-apochromatic 63x/1.4 NA objective lens (Zeiss) for PFA-fixed cells. Images were acquired and analyzed with the ZEN software (Zeiss).

PLA2-GIB enzymatic assay on [3H] arachidonic acid labelled CD4 T cells or Jurkat E6.1 T cells—Purified CD4 T-cells were incubated for 16 h at 2×10⁶ cells/ml with 1 μCi/ml of arachidonic acid [5,6,8,9,11,14,15-³H(N)] (Perkin Elmer, NET298Z250UC) in RPMI 1640 medium (Lonza) supplemented with 10% FBS, 50 mM HEPES pH 7.4, glutamine, penicillin, streptomycin and fungizone at 2 ml/well in 6-well plates at 37° C. in a 5% CO2 humidified atmosphere. Cells were washed twice with RPMI with 10% FBS by centrifugation at 580×g for 10 minutes at room temperature and then frozen in 90% FBS 10% DMSO at 10⁷ cells/ml/vial at −80° C. Percent of [3H] arachidonic acid in CD4 T cells is the (1 minus ratio of [3H] arachidonic acid in the supernatant of CD4 T cells without cells (cpm/ml) on total [3H] arachidonic acid in supernatant and cells (cpm/ml).

Jurkat E6.1 T cells (ECACC 88042803) or gC1qR KO Jurkat E6.1 T cells were incubated for 17 h at 5×10⁵ cells/ml with 1 μCi/ml of arachidonic acid [5,6,8,9,11,14,15-³H(N)] (Perkin Elmer, NET298Z250UC) in RPMI 1640 medium (Lonza) supplemented with 10% FBS, 50 mM HEPES pH 7.4, glutamine, penicillin, streptomycin and fungizone at 2 ml/well in 6-well plates at 37° C. in a 5% CO2 humidified atmosphere. Cells were washed twice with RPMI with 10% FBS by centrifugation at 300×g for 10 minutes at room temperature and then frozen in 90% FBS 10% DMSO at 10⁷ cells/ml/vial at −80° C. Percent of [3H] arachidonic acid in CD4 T cells is the (1 minus ratio of [3H] arachidonic acid in the supernatant of CD4 T cells without cells (cpm/ml) on total [3H] arachidonic acid in supernatant and cells (cpm/ml).

To test PLA2-GIB activity on [3H] arachidonic acid labelled CD4 T lymphocytes, cells were unfrozen in 10% FBS RPMI preheated at 37° C. by centrifugation at 580×g for 10 minutes at room temperature, washed twice in 2.5% FBS RPMI, and equilibrated at 2×10⁵ CD4 T cells/400 μl/well in 24-well polystyrene plates for 1 h30 at 37° C. in a 5% CO2 humidified atmosphere. Then 100 μl of recombinant proteins (gp41 MN (565-771Delta642-725), Antibodies online, ABIN2129703; HCV core protein, HCV-011, Prospec) or vehicle dilution in 2.5% FBS RPMI was added to each well for 2 h. Cells and supernatant were collected in eppendorf tubes and centrifuged at 580×g for 10 minutes at room temperature. The [3H] arachidonic acid released in cell supernatant was quantified in 300 μl with 16 ml of Ultima gold (Perkin Elmer, 6013329) in low diffusion vials (Perkin Elmer, 6000477) on a counter (tri-Carb 2800 TR liquid scintillation analyzer, Perkin Elmer).

To test PLA2-GIB activity on [3H] arachidonic acid labelled Jurkat E6.1 T lymphocytes, cells were unfrozen in 10% FBS RPMI preheated at 37° C. by centrifugation at 300×g for 10 minutes at room temperature, washed twice in 2.5% FBS RPMI, and equilibrated at 5×10⁴ or 10⁵ Jurkat E6.1 T cells/400 μl/well in 24-well polystyrene plates for 1 h30 at 37° C. in a 5% CO2 humidified atmosphere. Then HCV core solution or vehicle dilution in 2.5% FBS RPMI at 5.95 μM was mixed with an equal volume of a PLA2GIB solution at 630 nM or 2 μM 2.5% FBS RPMI and 100 μl were added per well at the same time for 2 h. For peptide treatments, cells were pretreated for 2 h, 4 h or 21 h, as indicated on figures, with 50 μl per well of peptide solutions at 110 μM or 55 μM in 2.5% FBS RPMI. Then 50 μl per well of PLA2-GIB at 630 nM or 2 μM 2.5% FBS RPMI or medium alone were added for 2 h. Cells and supernatant were collected in eppendorf tubes and centrifuged at 580×g for 10 minutes at room temperature. The [3H] arachidonic acid released in cell supernatant was quantified in 300 μl with 16 ml of Ultima gold (Perkin Elmer, 6013329) in low diffusion vials (Perkin Elmer, 6000477) on a counter (tri-Carb 2800 TR liquid scintillation analyzer, Perkin Elmer).

Results are expressed as PLA2GIB activity (release of [3H] arachidonic acid in the supernatant of cells treated with peptide or HCV core together with PLA2-GIB minus spontaneous release of [3H] arachidonic acid by cells with peptide or buffer only without PLA2-GIB in cpm/ml) or ΔPLA2-GIB activity with peptides minus activity with Scrambled PEP3 (release of [3H] arachidonic acid in the supernatant of cells treated with peptide minus release of [3H] arachidonic acid by cells treated with Scrambled PEP3 in cpm/ml).

### Results and Discussion

### 1. Viremic Patient Plasma Increases the Activity of PLA2-GIB on CD4 T Cells

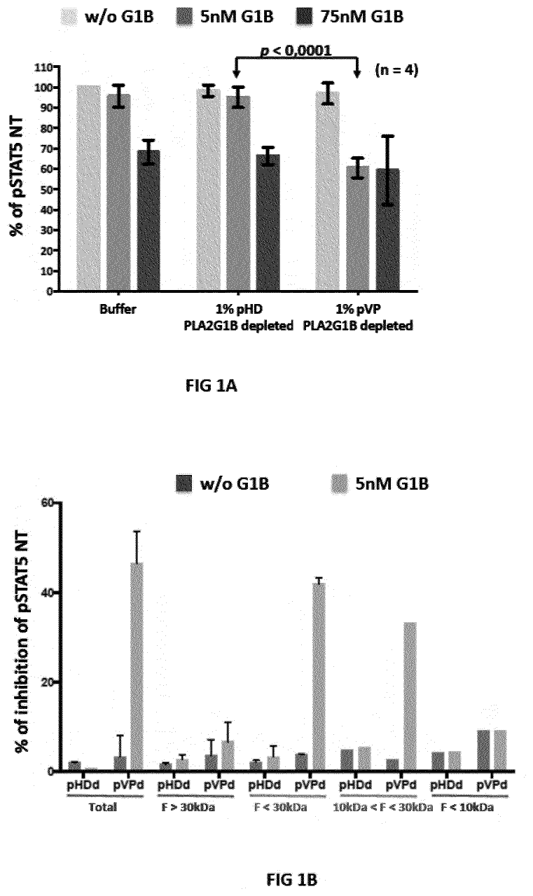

We have shown previously that treatment of CD4 T cells with 75 nM of PLA2-GIB alone significantly decreases the nuclear translocation of phosphoSTAT5 (pSTAT5 NT) induced by IL-7 while treatment with 5 nM of PLA2-GIB does not affect this response to IL-7 (Buffer, FIG. 1A). The endogenous PLA2-GIB-depleted viremic plasma does not affect phosphosSTAT5 translocation in response to IL-7. Notably addition of 5 nM of PLA2-GIB in 1% of endogenous PLA2-GIB-depleted viremic plasma results in 40% of inhibition pSTAT5 NT while healthy donor plasma similarly treated has no effect (n=4 independent donors, p<0.0001, FIG. 1A). These results demonstrate that viremic plasma contains a cofactor that sensitizes CD4 T cells to inhibition by PLA2-GIB.

To identify the molecular weight of this viremic plasma cofactor we fractionated viremic plasma on filter with 30 kDa and 10 kDa cut-off. As shown on FIG. 1B, the fraction of endogenous PLA2-GIB-depleted viremic plasma which contains products of more than 10 kDa and less than 30 kDa increases PLA2-GIB activity on CD4 T cells but not the same fraction from healthy donor plasma. The fraction of endogenous PLA2-GIB-depleted viremic plasma which contains products of more than 30 kDa and the fraction which contains products of less than 10 kDa have no effect on PLA2-GIB activity. Thus, viremic plasma patient contains a cofactor with a molecular weight between 10 kDa and 30 kDa that sensitizes CD4 T cells to inhibition by PLA2-GIB under experimental conditions where PLA2-GIB concentration alone is not sufficient to affect pSTAT5 NT in response to IL-7.

### 2. HIV-1 Inactivated Viral Particles Sensitize CD4 T Cells to PLA2-GIB Inhibitory Activity on Response to IL-7

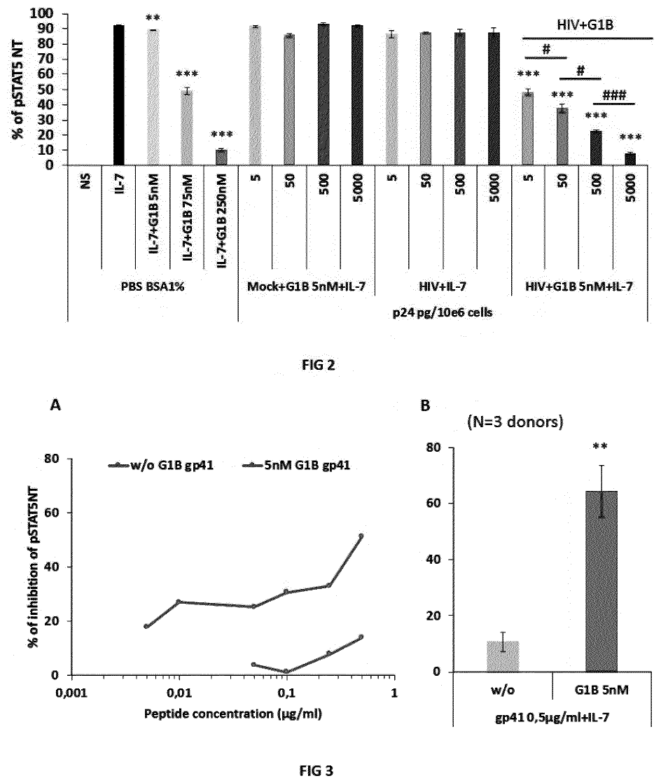

To test the hypothesis that HIV-1 viral products could play a role in the cofactor activity of viremic plasma we first investigated the effect of HIV-1 particles on pSTAT5 NT response to IL-7 in healthy donor CD4 T cells. We used HIV-1 particle of a T-tropic HIV-1 NDK virus previously inactivated with AT-2 to test the effect of viral proteins on CD4 T cells in absence of infection. To test the cofactor activity, CD4 T cells were exposed to different amount of HIV particles (MOI 1, 0.1, 0.01 and 0.001) alone or in presence to an amount of PLA2-GIB (5 nM) that does not inhibit phosphoSTAT5 nuclear translocation in response to IL-7 (FIG. 2). pSTAT5 NT in response to IL-7 was more than 92% without PLA2-GIB, biologically similar with 5 nM of PLA2-GIB and only at 50% and 10% with 75 nM and 250 nM of PLA2-GIB as expected. HIV-1 particles alone do not affect pSTAT5 NT in response to IL-7. Of note HIV-1 particles results in a dose-response inhibition of pSTAT5 NT in presence of 5 nM of PLA2-GIB (48% of pSTAT5 NT with 5pg/ml of p24 (MOI=0.001) to only 8% of pSTAT5 NT with an 5000pg/ml of p24 (MOI=1), p<0.001, FIG. 2) while similar dilutions of control (Mock) with 5 nM of PLA2-GIB have no effect on pSTAT5 NT in response to IL-7. These results demonstrate that some viral components could play the role of cofactor that sensitize CD4 T cells to PLA2-GIB activity as observed in viremic patient plasma.

3. HIV-1 gp41 Protein Increases PLA2-GIB Inhibitory Activity on pSTAT5 NT in CD4 T Cells Stimulated with IL-7

We analyzed pSTAT5 NT response to IL-7 in cells pretreated with a dose of PLA2-GIB that cannot inhibit pSTAT5 NT in absence of cofactor (5 nM), together or not with a recombinant gp41 protein or with gp41 protein alone, without PLA2-GIB (w/o GIB). As shown on FIG. 3, gp41 protein alone as a minor inhibitory effect on pSTAT5 NT response to IL-7 at 0.5 μg/ml of gp41 with only 10% of inhibition and less than 8% of inhibition with 0.25 to 0.05 μg/ml of gp41 (FIGS. 3A and 3B). By a striking contrast, in presence of 5 nM of PLA2-GIB, 0.5 μg/ml of gp41 protein resulted in more than 60% of inhibition of pSTAT5 NT (FIG. 3B) with a dose-dependent inhibition to 18% of inhibition with 0.005 μg/ml of gp41 (FIG. 3A).

4. HIV-1 gp41 Protein Plays a Critical Role in the Inhibitory Activity of Viremic Patient Plasma on pSTAT5 NT in CD4 T Cells Stimulated with IL-7

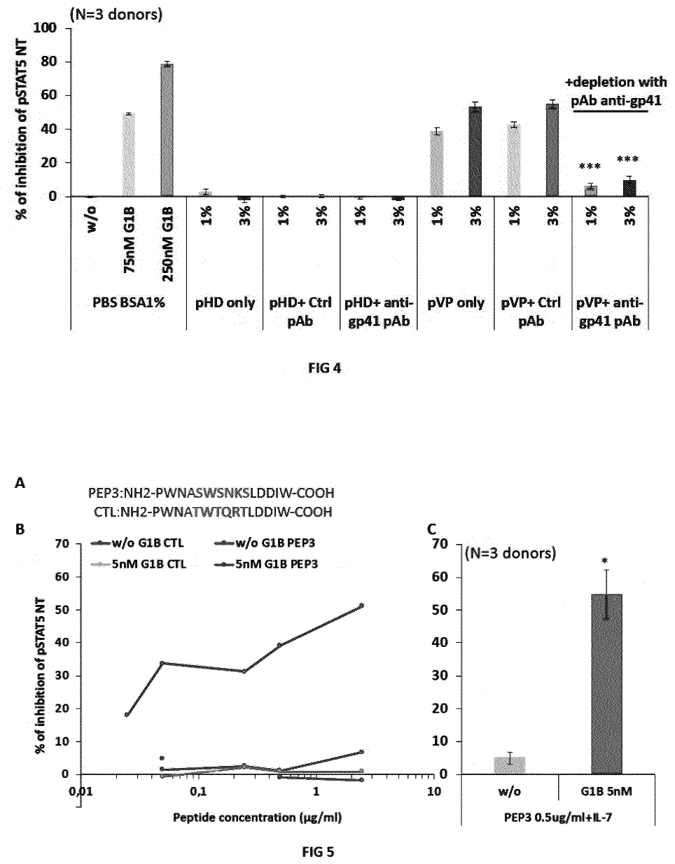

To verify that gp41 protein could be a cofactor of PLA2-GIB in viremic patient plasma, we depleted viremic patient plasma with polyclonal antibody against gp41 (pAb anti-gp41) or control polyclonal antibody (pAb ctrl). Healthy donor plasma was similarly treated as negative control. As presented on FIG. 4, the inhibition of pSTAT5 NT was 49% with 75 nM and 79% with 250 nM of PLA2-GIB as expected and 39% with 1% and 54% with 3% of viremic patient plasma without antibody. Healthy donor plasma had no inhibitory effect on pSTAT5 NT in response to IL-7 without antibody, with control polyclonal antibody or anti-gp41 polyclonal antibody which demonstrates that antibodies have no toxicity on CD4 T cells (FIG. 4). Treatment of viremic patient plasma with control polyclonal antibody does not change the inhibitory activity with 43% and 55% of inhibition with 1% and 3% of plasma respectively (FIG. 4). Notably, immunodepletion with anti-gp41 polyclonal antibody almost abrogated the inhibitory activity of viremic patient plasma with 6% and 10% of residual inhibitory activity with 1% and 3% of immunodepleted plasma (p<0.001 pAb anti-gp41 vs pAb ctrl treated plasma, FIG. 4). Altogether these results demonstrate that gp41 is a cofactor of PLA2-GIB in viremic patient plasma.

5. The PEP3 Motif in Gp41 Inhibits pSTAT5 NT in CD4 T Cells Stimulated with IL-7

CD4 T cells were exposed to a 15 aminoacids peptide domain of gp41 containing a potential gC1qR binding element. The peptide contains SWSNKS motif. The cells were also exposed to a control (CTL) peptide (FIG. 5A), together with 5 nM of PLA2-GIB (5 nM GIB) or not (w/o). While CTL peptide alone or with PLA2-GIB or PEP3 alone have no effect on pSTAT5 NT, treatments with PEP3 and 5 nM of PLA2-GIB resulted in a PEP3 dose-dependent inhibition of pSTAT5 NT from 51% to 18% of inhibition with 2.5 μg/ml to 0.025 μg/ml of PEP3 respectively (FIG. 5B). As summarized on FIG. 5C, treatment with 0.5 μg/ml of PEP3 alone only resulted in 5% of inhibition of pSTAT5 NT in CD4 T cells while treatment with 0.5 μg/ml of PEP3 together with 5 nM PLA2-GIB resulted in 55% of inhibition (p<0.05, n=3 donors).

### 6. PEP3 has a Cofactor Effect on PLA2GIB

PEP3 effect on PLA2-GIB activity was assessed on [3H] AA Jurkat E6.1 T cells. 5×10⁴ cells Jurkat E6.1 were pretreated with PEP3 or scrambled PEP3 for different periods of time (up to 21 hours). 2 h post-treatment, the cells were incubated with 200 nM PLA2-GIB. The results are presented on FIG. 11 (pool of 3 experiments).

As can be seen, pretreatment of cells 4 h or more with peptide PEP3 peptide (11 μM) significantly increased PLA2-GIB activity on the membrane of Jurkat E6.1 T cells vs scrambled PEP3 (p<0.001). These results confirm that PEP3 has a cofactor effect on PLA2GIB.

7. The Cofactor Activity of PEP3 on PLA2-GIB and the Inhibitory Activity of Viremic Patient Plasma are Dependent on gC1qR

We hypothesized that gC1qR could play a role in the inhibitory activity of viremic patient plasma. To study the role of gC1qR in PLA2-GIB inhibition of pSTAT5 NT, we tested the effect of C1q, the natural ligand of gC1qR, on PLA2-GIB activity. We found that C1q alone was able to inhibit 40% pSTAT5 NT (p<0.001). PLA2-GIB addition to C1q increases this inhibitory activity to 75-85% of inhibition, (p<0.01, FIG. 6A) and C1q effect as well as cofactor effect on PLA2-GIB was significantly inhibited with two different anti-gC1qR antibodies that restore 75% of response (60.11 and 75.4.2 anti-gC1qR antibodies vs IgG1ctrl with C1q and 5 nM PLA2-GIB, p<0.001, FIG. 6A). Notably the anti-gC1qR antibody 74.5.2 restore 54% of pSTAT5 NT in presence of PEP3 and PLA2-GIB (FIG. 6B, p<0.0001) and 32% of pSTAT5 NT in cells treated with 1% of viremic patient plasma (FIG. 6C, p<0.0001). By contrast, the control antibody (IgG1 ctrl) does not inhibit PEP3 cofactor activity on PLA2-GIB nor viremic patient plasma effect (FIGS. 6B and 6C).

8. PEP3 Binds to gC1qR

The binding of PEP3 to gC1qR was tested by ELISA assay on microplates as described in the materials and methods. A scrambled peptide or a control peptide were used as control.

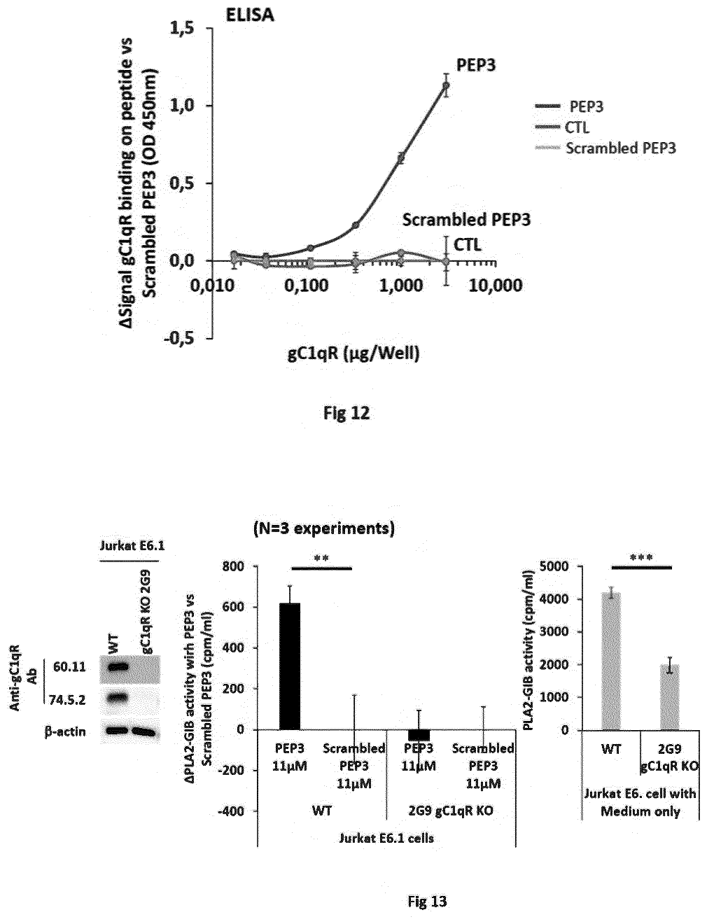

The results are presented on FIG. 12. They show that PEP3 binds to gC1qR while the scrambled and control peptides essentially do not.

9. gC1qR is Involved in PEP3 Cofactor Effect on Jurkat T Cells Membranes

The effect of PEP3 and gC1qR on PLA2-GIB activity was tested on [3H] AA Jurkat E6.1 cells. 5×10⁴ cells Jurkat E6.1 WT, gC1qR KO (1D5 or 2G9), were pretreated for 21 h with PEP3 or scrambled PEP3. 2 h post-treatment, the cells were incubated with PLA2-GIB.

The results are presented on FIG. 13 (pool of 3 experiments). Pretreatment 21 h of WT cells but not gC1qR KO cells with PEP3 peptide increased significantly PLA2-GIB activity vs scrambled PEP3 (p<0.01). The PLA2-GIB activity is significantly higher on WT than gC1qR KO cells.

These results further show that gC1qR is involved in PEP3 cofactor effect.

### 10. Gp41 Protein Sensitizes CD4 T Cells Membranes to PLA2-GIB Enzymatic Activity

To study PLA2-GIB effect on CD4 T cells membranes we developed a new enzymatic assay in which CD4 T cells are labelled with [3H] arachidonic acid. When these cells are exposed to PLA2-GIB the enzymatic activity on CD4 T cells releases [3H] arachidonic acid. The quantification of [3H] arachidonic acid allowed us to quantify PLA2-GIB activity.

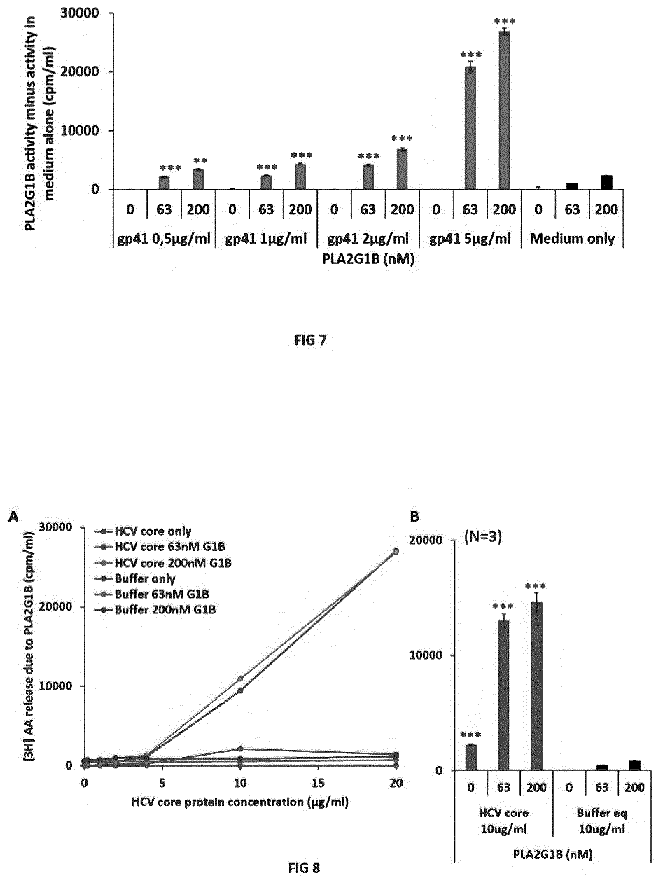

As we observed above that gp41 protein can increase PLA2-GIB inhibitory activity on pSTAT5 NT, we postulated that gp41 could increase PLA2-GIB enzymatic activity on CD4 T cells membranes. Indeed, PLA2-GIB enzymatic activity is highly and significantly increased when gp41 is present and in a gp41 dose-dependent manner (p<0.01 and p<0.001, FIG. 7). Gp41 treatment alone has no effect on [3H] arachidonic acid release by CD4 T cells. Treatments with 0.5 to 5 μg/ml of gp41 resulted in a 2.2 to 21-fold increase of 63 nM of PLA2-GIB activity and 1.5 to 11.6-fold increase of 200 nM of PLA2-GIB activity on [3H] arachidonic acid release by CD4 T cells with a maximum at 5 μg/ml of gp41. Treatment with 5 μg/ml of gp41 can increase the activity of PLA2-GIB more than 70-Fold on some donor.

### 11. Other PLA2-GIB Cofactors

Our demonstration that gC1qR is a sensor of PLA2-GIB cofactor led us to investigate other gC1qR ligands.

Table 2 lists 30 different molecules that bind to gC1qR and can thus affect PLA2-GIB activity. About half of these molecules are derived from pathogens: 9 are viral proteins, 4 are bacterial components and one is the Plasmodium falciparum parasite (Table 2). One molecule, LyP-1, is an artificial gC1qR ligand and the other 15 are endogenous components, five from serum and 10 from cells. Altogether these results suggest that PLA2-GIB activity can be modulated by various distinct pathogen components and endogenous factors, and that this pathway is a general mechanism of pathogenesis.

### 12. HCV Core Protein Sensitizes CD4 T Cells Membranes to PLA2-GIB Enzymatic Activity

We analyzed PLA2-GIB enzymatic activity on CD4 T cells in the presence of recombinant HCV core protein (FIG. 8). HCV core protein contains a gC1qR binding element (see table 2). Our results show that HCV core protein alone slightly induces the release of [3H] arachidonic acids by CD4 T cells at 10 and 20 μg/ml (FIG. 8A). Interestingly treatments of CD4 T cells with PLA2-GIB and HCV core protein highly increases PLA2-GIB enzymatic activity with a 26-fold and 36-fold increase of activity of 63 nM of PLA2-GIB and 16-fold and 26-fold increase of activity of 200 nM of PLA2-GIB at 10 and 20 μg/ml of HCV core protein (FIG. 8A). As summarized on FIG. 8B, treatment with 10 μg/ml of HCV core protein alone slightly and significantly increases the release of [3H] arachidonic acids by CD4 T cells. Furthermore, HCV core protein is a very potent PLA2-GIB cofactor with 26-fold and 16-fold increase activity of 63 nM and 200 nM of PLA2-GIB respectively (p<0.001, n=3 donors). These results show that HCV core protein can sensitize CD4 T cells to PLA2-GIB inhibition, thus leading to an inhibition of CD4 T cells function in patients with hepatitis C infection.

### 13. HCV Core Protein Sensitizes Jurkat E6.1 T Cells Membranes to PLA2-GIB Enzymatic Activity

HCV core protein effect on PLA2-GIB activity was further tested on Jurkat E6.1 cells. HCV core (595 nM equivalent as 10 μg/ml) was incubated with 5×10e⁴ cells. The release of [3H] AA due to PLA2-GIB minus activity in eq buffer was measured.

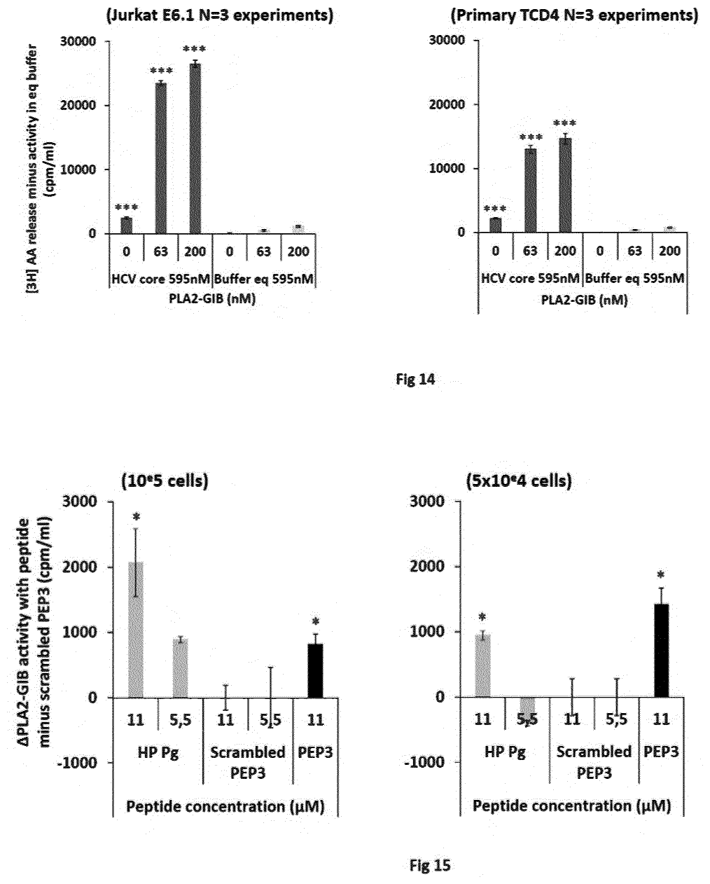

The results are presented on FIG. 14. They show that HCV core protein significantly increased PLA2-GIB activity on the membrane of Jurkat E6.1 T cells similarly as observed on CD4 T cells membrane.

HCV core protein thus exhibit potent cofactor effect.

14. Staphylococcus aureus Protein a (SA Protein A) Sensitizes CD4 T Cells to PLA2-GIB Enzymatic Activity

We analyzed the effect of the SA protein A, another gC1qR binding protein (Table 2), on PLA2-GIB enzymatic activity on CD4 T cells (FIG. 9). As observed with HCV core protein, SA protein A alone slightly induces the release of [3H] arachidonic acids by CD4 T cells at 10, 25 and 50 μg/ml (FIG. 9A, p<0.01). Notably treatments of CD4 T cells with PLA2-GIB and SA protein A significantly increases PLA2-GIB enzymatic activity 1.5-fold to 3-fold more activity of 200 nM of PLA2-GIB at 10 to 50 μg/ml of SA protein A (FIG. 9B, p<0.0001). These results show that SA protein A can sensitize CD4 T cells to PLA2-GIB inhibition, thus leading to an inhibition of CD4 T cells function in patients with Staphylococcus aureus infection. These results also complete the above observation with the viral protein HCV core and demonstrate that bacteria proteins that binds to gC1qR could be PLA2-GIB cofactors. Altogether HCV core protein and SA protein A experiments suggest that gC1qR activation/PLA2-GIB sensitization could be a general mechanism by which pathogens act.

15. Identification of gC1qR-Binding Domain-Containing Proteins that can Act as PLA2-GIB Cofactors

We screened protein database with PEP3 peptide sequence to identify other proteins containing a gC1qR-binding element. 42 Proteins from 27 different bacteria species and one fungus (Candida glabrata) were identified, one was from ananas, another from Caenorhabditis elegans and the last one was from human (Table 1). Among them, we identified 11 proteins from 9 human pathogens (8 bacteria and 1 fungus) that could regulate PLA2-GIB activity, as summarized in Table 3. These pathogens have been associated with cancer, autoimmune and neurodegenerative diseases. For instance, Porphyromonas gingivalis infection is associated with pancreatic cancer, Rheumatoid arthritis, Alzheimer's disease and Candida glabrata infection is associated with cutaneous candidiasis in HIV/AIDS patients, patients with cancer and chemotherapy treatment and organ transplantation.

### 16. HP Porphyromonas Gingivalis PEPTIDE 8 (HP Pg) has a Cofactor Effect on PLA2-GIB

The effect of peptide HP Pg on the activity of PLA2-GIB was measured on [3H] AA Jurkat E6.1 cells. 10e⁵ (left panel) or 5×10e⁴ (right panel) cells Jurkat E6.1, were pretreated 21 h with HP Pg, scrambled PEP3 or 3S. 2 h post-treatment, the cells were incubated with 200 nM PLA2-GIB.

The results are presented FIG. 15. They show that pretreatment with HP Pg (SEQ ID NO: 8) significantly increased PLA2-GIB activity vs scrambled PEP3.

These results demonstrate that HP Pg has a cofactor effect.

### 17. PDAC Plasma has a Cofactor Effect on PLA2GIB

We tested the capacity of the plasma from PDAC patients to modulate IL-2 response of CD4 T cells by measuring the phospho-STAT5 nuclear translocation (pSTAT5 NT).

As shown in FIG. 16, we observed an inhibitory effect of PDAC plasma on CD4 T cell IL-2 response at 1% and 3% dilution. This result demonstrates that, in cancer patients, the tumor microenvironment or plasma provides immune modulation, e.g. inhibition. This finding indicates that cancers contain a PLA2-GIB cofactor which renders T cells sensitive to inactivation by PLA2-GIB.

TABLE 1

| | ACCESSION | | SEQUENCE OF gC1qR | SEQ ID |
|---|---|---|---|---|
| PROTEIN NAME | NUMBER | SPECIES | BINDING ELEMENT | NO: |
| gp41 | AAC31817.1 | HIV | PWNASWSNKSLDDIW | 3 |
| | | | | (residues |
| | | | | 97-111) |
| UNKNOWN | WP_077094164.1 | Porphyromonas gingivalis | AWNAIWINRKYEQID | 4 |
| UDP-glucose 4-epimerase | SJM20449.1 | Porphyromonas gingivalis | GIAESWPNSLDDSCA | 5 |
| L-threonine 3-dehydrogenase | WP_013815975.1 | Porphyromonas gingivalis | GIAESWPNSLDDSCA | 6 |
| TonB-dependent receptor | WP_097552718.1 | Porphyromonas gingivalis | SFLKSWFNNSLVDIG | 7 |
| UNKNOWN | WP_097552551.1 | Porphyromonas gingivalis | SGEGGWSNGSLVDIM | 8 |
| DNA polymerase III subunit | SJM20595.1 | Porphyromonas gingivalis | YELDAASNNSVDDIR | 9 |
| gamma/tau | | | | |
| Multidrug transporter AcrB | WP_097555277.1 | Porphyromonas gingivalis | AALGKTLVKSLDDIP | 10 |
| Peptide ABC transporter permease | AIF49259.1 | Dyella japonica | PWNASWSDKFYENSL | 11 |
| Peptide ABC transporter | WP_019421834.1 | Paenibacillus sp. | AIHASWSNTSYEVID | 12 |
| substrate binding protein | | | | |
| Peptide ABC transporter | OJX83063.1 | Mesorhizobium sp. | PWNAGWSNARFDELC | 13 |
| substrate binding protein | | | | |
| MC73_05565 | KGY43685.1 | Proteus mirabilis | PWNAIWSAKNTTVDS | 14 |
| Chitinase | WP_068978097.1 | Aeromonas sp. | PWNASWSAAGVGAHA | 15 |
| TonB-dependent receptor | KZY48959.1 | Pseudoalteromonas sp. | WFNASWKDKSYSTVW | 16 |
| TonB-dependent receptor | WP_036212264.1 | Lysobacter arseniciresistens | PWNASWSVRHISELE | 17 |
| LVIVD repeat protein | EMY15726.1 | Leptospira weilii str. | PWNASWSYVLDSAWS | 18 |
| AMK72_12855 | KPK43807.1 | Planctomycetes bacterium | PWNGSWSNDAWGPGT | 19 |
| Peptide ABC transporter | OJX83063.1 | Mesorhizobium sp. | PWNAGWSNARFDELC | 20 |
| substrate-binding protein | | | | |
| UNKNOWN | WP_030088081.1 | Streptomyces baarnensis | PWNAGWSLKSSGKSA | 21 |
| HMPREF0183_2345 | EFG46376.1 | Brevibacterium mcbrellneri | PWNAWWSNRSMIADV | 22 |
| UNKNOWN | WP_048669463 | Vibrio crassostreae | AWNESWSNKSFHNGA | 23 |
| UNKNOWN | WP_070707834.1 | Porphyromonas sp. | EFNANWSNKFYLYNQ | 24 |
| | | HMSC077F02 | | |
| FAD-linked oxidoreductase | WP_084705378.1 | Leucobacter chironomi | RWNTSWSNWARTERS | 25 |
| RNA-binding protein 48 | XP_020288140.1 | Pseudomyrmex gracilis | NWNTSWSNTASGSDS | 26 |
| TonB-dependent receptor | WP_083710929.1 | Proteiniphilum | SINAAWSNQSYGFSR | 27 |
| | | saccharofermentans | | |
| Peptide ABC transporter | WP_074918487.1 | Terrisporobacter glycolicus | NNNAQWSNKEYDKIV | 28 |
| Type IV secretion protein Rhs | WP_032559173.1 | Bacteroides fragilis | HTCASWCNKSLSDIV | 29 |
| Putative transmembrane rhomboid | CAH09895.1 | Bacteroides fragilis | KDITSWVNKALDAIA | 30 |
| family protein | | | | |
| Receptor kinase-like protein Xa21 | XP_020096846.1 | Ananas comosus | GALSSWSNKSLHCCE | 31 |
| Rim ABC transporter | AAC05632.1 | Homo sapiens | EKVANWSIKSLGLTV | 32 |
| Importin beta SMX1 | KTB14942.1 | Candida glabrata | KFIESWSNKSLWLGE | 33 |
| Transporter | WP_049174838.1 | Acinetobacter ursingii | FLLYALSNKSLNDIW | 34 |
| Sugar ABC transporter permease | WP_075333493.1 | Pseudonocardia sp. | PTSVSWSNYEQILVG | 35 |
| ABC transporter substrate | WP_009846178.1 | Vibrio sp. | DEQKQWRNKSLEQLW | 36 |
| binding protein | | | | |
| Tetraspanin | NP_001024415.2 | Caenorhabditis elegans | YLGVSWSNKSLLYSY | 37 |
| Nucleotidyltransferase | WP_061886850.1 | Aggregatibacter | MSKFGLSDKSIEQIH | 38 |
| | | actinomycetemcomitans | | |
| Phospholipase C, | WP_026813378.1 | Arenibacter certesii | MRYTVESGKSLDDIW | 39 |
| phosphocholine-specific | | | | |
| Response regulator of zinc | WP_087741064.1 | Proteus mirabilis | FELVCASNKSLEQLA | 40 |
| sigma-54-dependent | | | | |
| two-component system | | | | |
| UNKNOWN | WP_018028649.1 | Porphyromonas somerae | DHDKGLETESLEQIW | 41 |
| Peptide ABC transporter permease | WP_065295736.1 | Aggregatibacter aphrophilus | EPKDFRESATLNQIW | 42 |

TABLE 2

| Pathogen | Ligand | ID NO: |
|---|---|---|
| Virus | HIV gp41 | SEQ ID NO: 3 |
| | HIV rev protein | 51 |
| | HCV core protein (a.a 26-124) | SEQ ID NO: 43 |
| | EBV EBNA1 | 45 |
| | Adenovirus core protein V | 46 |
| | Hantaan virus (HTNV) capsid | 47 |
| | HSV Neurovirulence factor ICP34 | 48 |
| | Rubella virus protease p150 | 49 |
| | Rubella virus capsid protein | 50 |
| Bacteria | Staphylococcus aureus protein A | SEQ ID NO: 44 |
| | Intemalin InIB Listeria monocytogenes | 52 |
| | Streptococcus pneumoniae hyaluronate lyase | 53 |
| | Exosporium of Bacillus cereus | 54 |
| Parasite | Plasmodium falciparum | 55 |
| Artifical ligand | LyP-1 | 56 |
| Serum components | C1q | 57 |
| | Kininogen | 58 |
| | Vitronectin | 59 |
| | Hyaluronan | 60 |
| | Tissue factor pathway inhibitor-2 (TFPI-2) | 61 |
| Co-receptor | DC-SIGN (CD209) | 62 |
| Mitochondrial protein | Mitochondrial antiviral-signaling protein (MAVS) | 63 |
| Cytosol/nuclear | Nucleus-related like TFII B | 64 |
| | Laminin B receptor p58 | 65 |
| | splicing factor-2 (ASF/SF2) | 66 |
| | Cytokeratin 1 | 67 |
| | CDKN2A isoform smARF | 68 |
| | PPIF | 69 |
| | U2AF1L4 | 70 |
| | Nop52 | 71 |

TABLE 3

| PROTEIN | SEQUENCES | SEQ ID NO: | PATHOLOGY |
|---|---|---|---|
| TonB-dependent receptor | SFLKSWFNNSLVDIG | 7 | Pancreatic cancer, Chronic |
| UNKNOWN | SGEGGWSNGSLVDIM | 8 | periodontal disease Rheumatoid |
| | | | polyarthritis, Alzheimer's Disease |
| MC73_05565 | PWNAIWSAKNTTVDS | 14 | Urinary tract infections, |
| | | | Osteomyelitis in a HIV-patient |
| LVIVD repeat protein | PWNASWSYVLDSAWS | 18 | Leptospirosis |
| Peptide ABC transporter | NNNAQWSNKEYDKIV | 28 | Wounds infection |
| Type IV secretion protein | HTCASWCNKSLSDIV | 29 | Peritoneal infections, bacteremia, |
| RhS | | | subcutaneous abscesses or burns |
| putative transmembrane | KDITSWVNKALDAIA | 30 | |
| rhomboid family protein | | | |
| Importin beta SMX1 | KFIESWSNKSLWLGE | 33 | Cutaneous candidiasis in |
| | | | HIV/AIDS, cancer and organ |
| | | | transplantation |
| Nucleotidyltransferase | MSKFGLSDKSIEQIH | 38 | Agressive Periodontitis, Bacterial |
| | | | vaginosis, Endocarditis, |
| | | | actinomycosis, Rheumatoid |
| | | | arthritis |
| UNKNOWN | DHDKGLETESLEQIW | 41 | Chronic skin, soft tissue and bone |
| | | | infections |
| Petide ABC transporter | EPKDFRESATLNQIW | 42 | Endocarditis, brain abscesses, |
| permease | | | vertebral osteomyelitis and |
| | | | bacteremia |

### REFERENCES

Ghebrehiwet B, Jesty J, Vinayagasundaram R, Vinayagasundaram U, Ji Y, Valentino A, Tumma N, Hosszu K H, Peerschke E I. Targeting gC1qR domains for therapy against infection and inflammation. Adv Exp Med Biol. 2013; 735:97-110.
Rossio J L, Esser M T, Suryanarayana K, Schneider D K, Bess J W Jr, Vasquez G M, Wiltrout T A, Chertova E, Grimes M K, Sattentau Q, Arthur L O, Henderson L E, Lifson J D. Inactivation of human immunodeficiency virus type 1 infectivity with preservation of conformational and functional integrity of virion surface proteins. J Virol. 1998 October; 72(10): 7992-8001.
Platt E J, Wehrly K, Kuhmann S E, Chesebro B, Kabat D. Effects of CCR5 and CD4 cell surface concentrations on infections by macrophagetropic isolates of human immunodeficiency virus type 1. J Virol. 1998 April; 72(4):2855-64.
Vieillard V, Strominger J L, Debré P. N K cytotoxicity against CD4+ T cells during HIV-1 infection: A gp41 peptide induces expression of an NKp44 ligand. PNAS 2005 Aug. 2; 102(31): 10981-6.
Fausther-Bovendo H, Vieillard V, Sagan S, Bismuth G, Debré P. HIV gp41 engages gC1qR on CD4+ T cells to induce the expression of an NK ligand through the PIP3/H2O2 pathway. PLoS Pathog. 2010 Jul. 1; 6:e1000975.
Kittlesen D J, Chianese-Bullock K A, Yao Z Q, Braciale T J, Hahn Y S. Interaction between complement receptor gC1qR and hepatitis C virus core protein inhibits T-lymphocyte proliferation. J Clin Invest. 2000 November; 106(10):1239-49.
Large M K, Kittlesen D J, Hahn Y S. Suppression of host immune response by the core protein of hepatitis C virus: possible implications for hepatitis C virus persistence. J Immunol. 1999 Jan. 15; 162(2):931-8.

1-31. (canceled)

32. A method of treating cancer in a mammalian subject comprising administering an inhibitor of a PLA2-GIB cofactor to the mammalian subject.

33. The method according to claim 32, wherein the cancer is selected from pancreatic cancer, melanoma, lung, oesophageal or pharyngeal cancer, retinoblastoma, liver, breast, ovary, renal, gastric, duodenum, uterine, cervical, thyroid, bladder, prostate, bone, brain or colorectal cancer.

34. The method according to claim 33, wherein the cancer is pancreatic cancer and is selected from pancreatic adenocarcinoma, neuroendocrine tumor, intraductal papillary-mucinous neoplasama, mucinous cystic neoplasm, and serious cystic neoplasm.

35. The method according to claim 32, said method reducing the rate of cancer occurrence, reducing the rate of cancer progression, reducing or treating cancer metastasis, killing cancer cells, or for treating risk factors for cancer, oro-gastro-intestinal inflammations, infections or pancreatitis.

36. The method according to claim 32, wherein the PLA2-GIB cofactor is a ligand of gC1qR, a protein selected from the proteins of Table 1 or 2, or a gC1qR-binding element of such a protein, a component of a pathogen or a nutrient or a protein or peptide from a pathogen, or a viral or bacterial or fungal or parasite protein or peptide.

37. The method according to claim 32, wherein the inhibitor inhibits binding of the cofactor to gC1qR or inhibits expression of the cofactor.

38. The method according to claim 32, wherein the inhibitor is a compound which binds to gC1qR or to the cofactor, and inhibits a function of gC1qR, a peptide, a lipopeptide, a nucleic acid, a carbohydrate, or an antibody or a variant or fragment of an antibody.

39. The method according to claim 38, wherein the inhibitor is an antibody, or a variant or fragment thereof, which binds gC1qR or a protein selected from Table 1 or 2, and optionally inhibits binding of said protein to gC1qR.

40. The method according to claim 38, wherein the inhibitor is a peptide which binds gC1qR and inhibits binding to gC1qR of a protein selected from Table 1 or 2.

41. The method according to claim 32, wherein the inhibitor is an immunogen of the PLA2-GIB cofactor, which can induce antibodies to the cofactor.

42. The method according to claim 32, wherein the inhibitor is administered in combination with another drug or treatment for cancer.

43. The method according to claim 42, wherein the inhibitor is administered in combination with chemotherapy or hormonotherapy.

44. The method according to claim 42, wherein the inhibitor is administered in combination with radiotherapy, ultrasound therapy or nanoparticle therapy.

45. The method according to claim 42, wherein the inhibitor is administered in combination with check-point inhibitors, immunotherapy or anti-cancer vaccines.

46. The method according to claim 42, wherein the inhibitor is administered in combination with an inhibitor of PLA2-GIB.

47. The method according to claim 46, wherein the inhibitor of PLA2-GIB is an antagonist of PLA2-GIB

48. The method according to claim 32, wherein the inhibitor is administered prior to, during or after surgery for said cancer.
